(19) United States
(12) Patent Application Publication
(10) Pub. No.: US 2024/0191199 A1
(43) Pub. Date: Jun. 13, 2024

(54) Hydrogel Scaffolds And Uses Thereof

(71) Applicant: Nanyang Technological University, Singapore (SG)

(72) Inventors: Lay Poh TAN, Singapore (SG); Feng WEN, Singapore (SG); Si Han Jasmine SEAH, Singapore (SG)

(21) Appl. No.: 18/287,676

(22) PCT Filed: Apr. 20, 2022
(86) PCT No.: PCT/SG2022/050234

§ 371 Date: Oct. 20, 2023

(30) Foreign Application Priority Data
Apr. 20, 2021 (SG) 10202104006R

(51) Int. Cl. C12N 5/077

(52) U.S. Cl. CPC C12N 5/0658; C12N 2502/00; C12N 2506/1384; C12N 2533/54; C12N 2533/74; C12N 2537/10

(57) ABSTRACT

The present invention relates to hydrogel scaffold and the uses thereof. In a specific embodiment, the hydrogel scaffold comprises gelatin methacryloyl (GelMA), alginate and pectin, and one or more cell types. In another embodiment, the one or more cell types comprise stem cells and/or progenitor cells. The stem cells are adipose-derived stem cells (ADSCs) or mesenchymal stem cells. The present invention further relates to the method of fabricating the hydrogel scaffold as well as a method of myogenic differentiation with the hydrogel scaffold.

### CROSS-REFERENCE TO RELATED APPLICATIONS

This application claims the benefit of priority of Singapore patent application No. 10202104006R, filed 20 Apr. 2021, the contents of it being hereby incorporated by reference in its entirety for all purposes.

### FIELD OF THE INVENTION

The present invention generally relates to the field of biotechnology. In particular, the present invention relates to hydrogel scaffolds and uses thereof.

### BACKGROUND OF THE INVENTION

Scaffolds are used to support cell growth or cell differentiation, or a combination thereof. Presently, it is common to upscale the production of cells using scaffolds for different applications, including the production of artificial foods to achieve sustainable and healthier food sources. Presently, most scaffolds include artificial components such as synthetic polymers, which have low biocompatibility for cell encapsulation and growth, and have therefore limited use in different applications, for example, cell-based research, food production, medical or cosmetic applications. Also, the scaffolds break easily during production, resulting in low production efficiency of the scaffolds.

In addition, present scaffolds require the use of expensive and complex cell medium that consists of chemical and biological components such as growth factors or differentiation-inducing factors to grow cells. These cell culture methods are very expensive, resulting in low returns of investment for any cell-based application.

In view of the above problems, there is a need to provide alternative scaffolds, in particular hydrogel scaffolds, that do not break easily during production and can support cell growth and/or differentiation. There is also a need to provide an alternative method of fabricating the scaffolds.

### SUMMARY OF THE INVENTION

In one aspect, there is provided a hydrogel scaffold comprising:
about 3.0-10.0 w/v % gelatin methacryloyl (GelMA);
about 0.5-2.0 w/v % alginate;
about 0.5-2.0 w/v % pectin; and
one or more cell types.

In another aspect, there is provided a method of myogenic differentiation comprising culturing one or more cell types in the hydrogel scaffold as disclosed herein in cell culture media, wherein the cell culture media comprises a basal medium and a serum.

In another aspect, there is provided a method of fabricating a hydrogel scaffold, the method comprising:
a) preparing a solution comprising alginate and pectin;
b) treating the solution from step a) to a temperature of about 4-120° C.;
c) adding gelatin methacryloyl (GelMA) to the heat-treated solution from step b);
d) adding one or more cell types to solution from step c);
e) setting an injection rate of about 0.1-2.0 ml/min;
f) wet-spinning a hydrogel scaffold.

### BRIEF DESCRIPTION OF THE DRAWINGS

The invention will be better understood with reference to the detailed description when considered in conjunction with the non-limiting examples and the accompanying drawings, in which:

FIG. 1 shows photos of the morphology of pADSC from porcine adipose tissue. pADSC adhered and expanded after (A) 3 days, (B) 6 days and (C) 8 days seeding on a 75 cm² culture flask with a seeding density of 6,000 cells/cm². Scalebar=200 μm. FIG. 1 shows that pADSCs have a fibroblast-like morphology.

FIG. 2 is a graph that shows the growth kinetics of pADSC harvested from porcine adipose tissue. FIG. 2 shows that pADSCs have a higher doubling time in the later passages (P6-P7).

FIG. 3 are column graphs to show the measurement of viscosities of polymer solutions. (A) viscosities of different polymers solutions (P1, P2 and P3) measured at different temperatures (20° C. and 37° C.) respectively. (B) viscosities of different polymers solutions (P1, P2 and P3) were measured at 37° C. before and after heat treatments (No Treatment; 80° C. for 2 hours; 120° C. for 20 mins). P1: 1% Alginate A+1% Pectin+3% GelMA; P2: 1% Alginate B+1% Pectin+3% GelMA; 1% Alginate C+1% Pectin+3% GelMA. FIG. 3 shows that heat treatment reduces the viscosity of the polymer mixtures.

FIG. 4 are column graphs to show the measurement of viscoelastic properties of heat-treated polymer solutions with shorter (6 min) or longer (10 min) duration of ultraviolet (UV) light exposure. Storage modulus (G′) and loss modulus (G″) of hydrogels formed were measured at 37° C. after PBS washing. Gel-P1H: UV-crosslinked hydrogel formed with P1 polymer solution after 80° C. heat treatment for 2 hrs; Gel-P2H: UV-crosslinked hydrogel formed with P2 polymer solution after 80° C. heat treatment for 2 hrs; Gel-P3H: UV-crosslinked hydrogel formed with P3 polymer solution after 80° C. heat treatment for 2 hrs. FIG. 4 shows that a longer ultraviolet (UV) light exposure time increases the stress and loss modulus of the polymer mixtures.

FIG. 5 is a schematic illustration of an exemplary experimental set-up for wet spinning and UV crosslinking.

FIG. 6 shows phase contrast images for pADSCs and C2C12 in the hydrogel scaffolds at different time points (Day 1, 3, 5 and 9). FIG. 6 shows that the cells were able to spread and proliferate in the hydrogel scaffolds.

FIG. 7 shows scanning electron microscopy (SEM) images Gel-P1H and Gel-P3H which are UV-crosslinked hydrogel scaffolds made from P1 and P3 polymer solutions respectively. Images in top panels: Scaffolds were washed with deionized water (DI) after wet spinning; Images in bottom panels: Scaffolds were washed with PBS after wet spinning. FIG. 7 shows the differences of the different hydrogel scaffolds made from different polymer solutions.

FIG. 8 shows fluorescent microscopy images of the hydrogel scaffolds after 9 days culture. Gel-P1H and Gel-P3H are UV-crosslinked hydrogel scaffolds made from heat-treated P1 and P3 polymers respectively. Strong calcein-AM positive signal revealed that most of cells were viable inside the hydrogel scaffolds and Ethidium Homodimer-1 (EthD-1) negative signals implied that there were negligible dead cells in the hydrogel scaffolds. FIG. 8 shows that the cells remain viable in the hydrogel scaffolds.

FIG. 9 shows SEM images for pADSCs and C2C12 cultured in hydrogel scaffolds after 9 days culture. Gel-P1H and Gel-P3H: Hydrogel scaffolds made from heat-treated P1 and P3 polymer solutions respectively. FIG. 9 shows the differences in morphology of the cells when encapsulated and cultured on the different hydrogel scaffolds made from different polymer solutions.

FIG. 10 shows fluorescent microscopy images of F-actin staining of cells (C2C12 and pADSC) encapsulated in hydrogel scaffolds after 9 days culture. FIG. 10 shows that the cells encapsulated and cultured on the hydrogel scaffolds formed tubular structures.

FIG. 11 shows fluorescent microscopy images of Anti-Myosin staining of cells (C2C12 and pADSC) encapsulated in hydrogel scaffolds after 9 days culture. Scalebar=100 μm. FIG. 11 shows that the cells encapsulated and cultured on the hydrogel scaffolds expressed a marker of skeletal muscle.

FIG. 12 shows fluorescent microscopy images of anti-YAP staining of pADSCs encapsulated in hydrogel scaffolds after 9 days culture. FIG. 12 shows that YAP is expressed in the cytoplasm when the cells encapsulated and cultured on the hydrogel scaffolds underwent myogenic differentiation.

FIG. 13 shows fluorescent microscopy images of C2C12 cells encapsulated in polymer with heat treatment (left) and polymer without heat treatment (right) on days 3 (top) and 9 (bottom), stained with calceinAM. FIG. 13 shows that the cells encapsulated and cultured on heat-treated hydrogel scaffolds were spread out. Scalebar=200 μm.

FIG. 14 shows images of calceinAM staining of C2C12 (top) and pADSCs (bottom) encapsulated in heat treated polymer, and cultured in growth media for 9 days. Scalebar=100 μm. FIG. 14 shows that the cells encapsulated and cultured on heat-treated hydrogel scaffolds were viable after 9 days of culture.

FIG. 15 shows images of wet-spun hydrogel scaffolds of polymer solutions with different pre-spinning treatments to show the difference in swelling over 14 days. Left column: polymer is treated at 80° C. for 1 hour followed by 4° C. incubation overnight. Right column: polymer is treated at 80° C. for 1 hour. FIG. 15 shows that the hydrogel scaffolds exhibited less swelling when the polymer is incubated 4° C. overnight. Scalebar=100 μm.

FIG. 16 shows images of pADSCs that have undergone adipogenesis, chondrogenesis and osteogenesis. Top left: The area circled by the white dashed line represents cells that are stained by Oil Red O solution, which represents cells that have undergone adipogenesis. Top right: The areas circled by the white dashed line represent cells that are stained by Toluidine Blue O solution, which represents cells that have undergone chondrogenesis. Bottom: The areas circled by the dashed line represent cells that are stained by Alizarin Red S solution, which represents cells that have undergone osteogenesis. FIG. 16 shows that the pADSCs are multipotent and can be differentiated to the adipogenic, chondrogenic and osteogenic lineages. Scalebar=100 μm.

### DETAILED DESCRIPTION

Hydrogel scaffolds are commonly used to provide structural support for cells to adhere and grow on. In recent years, different hydrogel scaffolds have also been developed for stem cell research, either to allow stem cells to retain their stemness, or to facilitate the differentiation of stem cells into different lineages. This allows a wide array of applications for hydrogel scaffolds in different technical disciplines, for example, food production, cell-based research, medical application or cosmetic application.

Globally, different techniques are developed to sustainably scale up the production of cells using hydrogel scaffolds, for example, by creating hydrogel scaffolds that can contribute to the modulation of, for example, cellular behaviour, cell growth, stem cell differentiation. One such method includes the use of scaffolds to grow different cells in complex cell culture media, however this results in an increase in cost as it includes the use of expensive chemical and biological components such as growth factors or differentiation-inducing factors, which limits the commercialization of the end products, for example, artificial foods or medical products. In addition, the present methods of using scaffolds also include the use of intensive chemical and biological treatments, which can lead to food or medical safety issues.

In recent years, scaffolds using plant-derived components have been developed, however these plant-derived scaffolds break easily during production. Furthermore, cells encapsulated in these plant-derived scaffolds do not grow well, which makes it difficult to scale up the growth and production of artificial foods from these cells.

In view of the above problems, the inventors have developed a hydrogel scaffold that can support cell growth and allow cell differentiation in simple cell culture media. The hydrogel scaffold also does not break easily during production.

In one example, the hydrogel scaffold of the present disclosure comprises:
about 3.0-10.0 w/v % gelatin methacryloyl (GelMA);
about 0.5-2.0 w/v % alginate;
about 0.5-2.0 w/v % pectin; and
one or more cell types.

Hydrogel scaffolds having the above composition show increased resistance to breakage during production, which improves the production efficacy of such scaffolds and reduces wastage of the starting material. The increased resistance to breakage is also useful for different downstream applications, such as food production, cell-based research, medical application, or cosmetic application.

As used herein, the term “hydrogel” refers to a crosslinked polymeric network that can retain large amount of water. The polymer used to form the hydrogel can be natural, synthetic or a combination thereof. The hydrogel is subsequently subjected to a fabrication method, for example but not limited to, wet-spinning, dry-spinning, 3D-printing, solution casting, freeze-drying, to form a hydrogel scaffold.

As used herein, the term “hydrogel scaffold” refers to a hydrogel that has been subjected to a fabrication method, for example but not limited to, wet-spinning, dry-spinning, or 3D-printing. The hydrogel scaffold comprises well-defined 3-dimensional (3D) hierarchical structures, for example but not limited to, a fiber-like structure or a sheet-like structure. The hydrogel scaffold comprises a diameter of about 250-600 μm. In one example, the hydrogel scaffold comprises a diameter of, but is not limited to about 250-450 μm, about 400-600 μm, about 250-350 μm, about 300-400 μm, about 350-450 μm, about 400-500 μm, about 450-550 μm, about 500-600 μm, or about 250 μm, about 300 μm, about 350 μm, about 400 μm, about 450 μm, about 500 μm, about 550 μm, or about 600 μm.

The stiffness and strength of the hydrogel scaffold is dependent on the type and concentration of components used. One component of the hydrogel scaffold as disclosed herein is gelatin methacryloyl (GelMA). GelMA is produced through the reaction of gelatin with methacrylic anhydride, wherein the number of amino groups in gelatin are substituted by methacryloyl groups. Due to the presence of methacryloyl groups, solution of GelMA can be crosslinked covalently under UV light when a photoinitiator is added. As GelMA is modified from natural polymer gelatin, it retains the tri-amino acid sequence arginine-glycine-aspartic acid (RGD) sequences that gelatin contains to promote cell adhesion, as well as the matrix metalloproteinases (MMP) sequence. GelMA has a porous microstructure, which provides an optimal environment for encapsulated cells to grow in as it allows diffusion of nutrients oxygen and waste exchange between the culture medium and encapsulated cells. GelMA is also biodegradable. GelMA is similar to gelatin as its solid-liquid transition is modulated by temperature. In addition, physical and chemical properties of GelMA hydrogels can be tuned flexibly to meet the requirements of various applications, therefore it is used as a material for the hydrogel scaffold. In one example, the hydrogel scaffold comprises about 3.0-10.0 w/v % GelMA. In another example, the hydrogel scaffold comprises, but is not limited to about 3.0-6.0 w/v % GelMA, about 5.0-8.0 w/v % GelMA, about 7.0-10.0 w/v % GelMA, or about 3.0 w/v % GelMA, about 4.0 w/v % GelMA, about 5.0 w/v % GelMA, about 6.0 w/v % GelMA, about 7.0 w/v % GelMA, about 8.0 w/v % GelMA, about 9.0 w/v % GelMA, or about 10.0 w/v % GelMA. In a preferred example, the hydrogel scaffold comprises about 3.0 w/v % GelMA.

In addition to gelatin methacryloyl (GelMA), the hydrogel scaffold further comprises alginate. Alginate is a naturally occurring anionic polymer typically obtained from sea plant and has been investigated and used for many biomedical applications, due to its biocompatibility, low toxicity, relatively low cost, and fast gelation by addition of divalent cations such as Ca²⁺. In one example, the hydrogel scaffold comprises about 0.5-2.0 w/v % alginate. In another example, the hydrogel scaffold comprises, but is not limited to about 0.5-1.5 w/v % alginate, about 1.0-2.0 w/v % alginate, or about 0.5 w/v % alginate, about 0.6 w/v % alginate, about 0.7 w/v % alginate, about 0.8 w/v % alginate, about 0.9 w/v % alginate, about 1.0 w/v % alginate, about 1.1 w/v % alginate, about 1.2 w/v % alginate, about 1.3 w/v % alginate, about 1.4 w/v % alginate, about 1.5 w/v % alginate, about 1.6 w/v % alginate, about 1.7 w/v % alginate, about 1.8 w/v % alginate, about 1.9 w/v % alginate or about 2.0 w/v % alginate. In a preferred example, the hydrogel scaffold comprises about 1.0 w/v % alginate.

The hydrogel scaffold also further comprises pectin. Pectin is a natural constituent of plant cell walls, possessing high molecular weight, and hydrophilicity, making it an ideal candidate for hydrogel formation and subsequently 3D bioprinting. In this work, alginate was selected to maintain rigidity of the hydrogel scaffold spun in Ca²⁺ solution temporally. Pectin is recognized for their properties as a natural gelling agent and thickener in a wide variety of food products. In one example, the hydrogel scaffold comprises about 0.5-2.0 w/v % pectin. In another example, the hydrogel scaffold comprises, but is not limited to about 0.5-1.5 w/v % pectin, about 1.0-2.0 w/v % pectin, or about 0.5 w/v % pectin, about 0.6 w/v % pectin, about 0.7 w/v % pectin, about 0.8 w/v % pectin, about 0.9 w/v % pectin, about 1.0 w/v % pectin, about 1.1 w/v % pectin, about 1.2 w/v % pectin, about 1.3 w/v % pectin, about 1.4 w/v % pectin, about 1.5 w/v % pectin, about 1.6 w/v % pectin, about 1.7 w/v % pectin, about 1.8 w/v % pectin, about 1.9 w/v % pectin or about 2.0 w/v % pectin. In a preferred example, the hydrogel scaffold comprises about 1.0 w/v % pectin.

The hydrogel scaffold as disclosed herein can also be amended by adjusting the different parameters of GelMA, for example, the degree of methacryloyl substitution (DS) of GelMA. As used herein, the terms “degree of substitution”, “degree of methacryloyl substitution” or “DS” refer to the number of substituent groups attached per base unit, in this case, the number of available cross-linkable methacryloyl groups (substituent group) that can crosslink with a photoinitiator crosslinker (base unit), for example, Irgacure 2959 or Lithium Phenyl (2,4,6-Trimethylbenzoyl) Phosphinate (LAP). A higher degree of methacryloyation substitution indicates that there is more available cross-linkable methacryloyl groups to crosslink with the photoinitiator crosslinker. This results in a higher crosslinking density after UV irradiation and hence a stiffer and stronger hydrogel scaffold, thereby providing resistance to breakage during production.

In one example, the GelMA of the hydrogel scaffold comprises a degree of methacryloyl substitution (DS) of about 25-99%. In another example, the GelMA of the hydrogel scaffold comprises a degree of methacryloyl substitution (DS) that can be, but is not limited to about 25%-50%, about 45%-70%, about 65%-99%, or about 25%-35%, about 35%-45%, about 45%-55%, about 55%-65%, about 65%-75%, about 75%-85%, about 85%-95%, about 90%-99%. In another example, the GelMA of the hydrogel scaffold comprises a degree of methacryloyl substitution (DS) that can be, but is not limited to about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%. In a preferred example, the GelMA of the hydrogel scaffold comprises a degree of methacryloyl substitution (DS) that is about 50%.

GelMA with a higher degree of methacryloyl substitution (DS) results in a stiffer hydrogel scaffold. For example, GelMA with a DS of about 90% is stiffer than GelMA with a DS of about 40%. Stiffness can be measured by the storage modulus. As used herein, the term “storage modulus” refers to the measure of the amount of energy required to be put into a material in order to distort it. Storage modulus provides an indication of the stiffness of a solid material. A higher storage modulus indicates a stiffer material. In one example, the hydrogel scaffold comprises a storage modulus of about 1-7 kPa. In another example, the hydrogel scaffold comprises a storage modulus that can be, but is not limited to about 1-1.5 kPa, about 1.5-2 kPa, about 2-2.5 kPa, about 2.5-3 kPa, about 3-3.5 kPa, about 3.5-4 kPa, about 4-4.5 kPa, about 5-5.5 kPa, about 5.5-6 kPa, about 6-6.5 kPa, about 6.5-7 kPa, or about 1 kPa, about 1.5 kPa, about 2 kPa, about 2.5 kPa, about 3 kPa, about 3.5 kPa, about 4 kPa, about 4.5 kPa, about 5 kPa, about 5.5 kPa, about 6 kPa, about 6.5 kPa, or about 7 kPa. In a preferred example, the hydrogel scaffold has a storage modulus of about 6 kPa.

Stiffness can be measured by the loss modulus. As used herein, the term “loss modulus” refers to the measure of the amount of energy dissipation when a material is distorted. Loss modulus can also provide an indication of the stiffness of a solid material. A lower loss modulus indicates a stiffer material. In one example, the hydrogel scaffold comprises a storage modulus of about 50-650 Pa. In another example, the hydrogel scaffold comprises a loss modulus that can be, but is not limited to about 50-100 Pa, about 100-150 Pa, about 150-250 Pa, about 250-300 Pa, about 300-350 Pa, about 350-400 Pa, about 400-450 Pa, about 500-550 Pa, about 550-600 Pa, about 600-650 Pa, or about 50 Pa, about 100 Pa, about 150 Pa, about 200 Pa, about 250 Pa, about 300 Pa, about 350 Pa, about 400 Pa, about 450 Pa, about 500 Pa, about 550 Pa, about 600 Pa, or about 650 Pa. In a preferred example, the hydrogel scaffold has a storage modulus of about 600 Pa.

It can be appreciated that the hydrogel scaffold as disclosed herein is tunable by combining different parameters as disclosed herein. Such tunable properties allow the hydrogel scaffold to withstand breakage during production and to meet the requirements of the application in which the hydrogel scaffold is used for. In addition, hydrogel scaffold can be adjusted to create an environment that accommodates the type of cells that are to be encapsulated in the hydrogel scaffold.

The hydrogel scaffold can comprise one or more cell types. In one example, the one or more cell types comprise stem cells and/or progenitor cells.

As used herein, the term “stem cell” refers to a cell that is undifferentiated or is partially differentiated, and can differentiate into various types of cells and proliferate indefinitely to produce more of the same stem cell. In one example, the stem cell density of the hydrogel scaffold is, but is not limited to about 5×10⁵ cells/ml to 5×10⁶ cells/ml, about 5×10⁵ cells/ml to 9×10⁵ cells/ml, about 8×10⁵ cells/ml to 2×10⁶ cells/ml, about 1×10⁶ cells/ml to 5×10⁶ cells/ml, or about 5.0×10⁵ cells/ml, about 5.5×10⁵ cells/ml, about 6.0×10⁵ cells/ml, about 6.5×10⁵ cells/ml, about 7.0×10⁵ cells/ml, about 7.5×10⁵ cells/ml, about 8.0×10⁵ cells/ml, about 8.5×10⁵ cells/ml, about 9.0×10⁵ cells/ml, about 9.5×10⁵ cells/ml, about 1.0×10⁶ cells/ml, about 1.5×10⁶ cells/ml, about 2.0×10⁶ cells/ml, about 2.5×10⁶ cells/ml, about 3.0×10⁶ cells/ml, about 3.5×10⁶ cells/ml, about 4.0×10⁶ cells/ml, about 4.5×10⁶ cells/ml, about 5.0×10⁶ cells/ml. In another example, the hydrogel scaffold comprises 5×10⁵ cells/ml to 2×10⁶ cells/ml stem cells. In another example, the hydrogel scaffold comprises 1×10⁶ cells/ml stem cells.

In one example, the stem cells are adipose-derived stem cells (ADSCs) or mesenchymal stem cells. In one example, the adipose-derived stem cells (ADSCs) or mesenchymal stem cells density of the hydrogel scaffold is, but is not limited to about 5×10⁵ cells/ml to 5×10⁶ cells/ml, about 5×10⁵ cells/ml to 9×10⁵ cells/ml, about 8×10⁵ cells/ml to 2×10⁶ cells/ml, about 1×10⁶ cells/ml to 5×10⁶ cells/ml, or about 5.0×10⁵ cells/ml, about 5.5×10⁵ cells/ml, about 6.0×10⁵ cells/ml, about 6.5×10⁵ cells/ml, about 7.0×10⁵ cells/ml, about 7.5×10⁵ cells/ml, about 8.0×10⁵ cells/ml, about 8.5×10⁵ cells/ml, about 9.0×10⁵ cells/ml, about 9.5×10⁵ cells/ml, about 1.0×10⁶ cells/ml, about 1.5×10⁶ cells/ml, about 2.0×10⁶ cells/ml, about 2.5×10⁶ cells/ml, about 3.0×10⁶ cells/ml, about 3.5×10⁶ cells/ml, about 4.0×10⁶ cells/ml, about 4.5×10⁶ cells/ml, about 5.0×10⁶ cells/ml. In another example, the hydrogel scaffold comprises 5×10⁵ cells/ml to 2×10⁶ cells/ml adipose-derived stem cells (ADSCs) or mesenchymal stem cells. In another example, the hydrogel scaffold comprises 1×10⁶ cells/ml adipose-derived stem cells (ADSCs) or mesenchymal stem cells.

As used herein, the term “progenitor cell” refers to a cell that can differentiate into a specific type of cell. Progenitor cells is more specific than a stem cell as they are at a further stage of differentiation than a stem cell. In one example, the progenitor cell density of the hydrogel scaffold is, but is not limited to about 5×10⁵ cells/ml to 5×10⁶ cells/ml, about 5×10⁵ cells/ml to 9×10⁵ cells/ml, about 8×10⁵ cells/ml to 2×10⁶ cells/ml, about 1×10⁶ cells/ml to 5×10⁶ cells/ml, or about 5.0×10⁵ cells/ml, about 5.5×10⁵ cells/ml, about 6.0×10⁵ cells/ml, about 6.5×10⁵ cells/ml, about 7.0×10⁵ cells/ml, about 7.5×10⁵ cells/ml, about 8.0×10⁵ cells/ml, about 8.5×10⁵ cells/ml, about 9.0×10⁵ cells/ml, about 9.5×10⁵ cells/ml, about 1.0×10⁶ cells/ml, about 1.5×10⁶ cells/ml, about 2.0×10⁶ cells/ml, about 2.5×10⁶ cells/ml, about 3.0×10⁶ cells/ml, about 3.5×10⁶ cells/ml, about 4.0×10⁶ cells/ml, about 4.5×10⁶ cells/ml, about 5.0×10⁶ cells/ml. In another example, the hydrogel scaffold comprises 5×10⁵ cells/ml to 2×10⁶ cells/ml progenitor cells. In another example, the hydrogel scaffold comprises 1×10⁶ cells/ml progenitor cells.

In one example, the progenitor cells are myoblasts or myosatellite cells. As used herein, the term “myosatellite cell” refers to a type of muscle progenitor cell. The myosatellite cell is usually quiescent and do not undergo cell proliferation or cell differentiation when they are not activated. When the myosatellite cell is activated by an external factor, for example, mechanical stimulation, the myosatellite cell is activated and undergoes cell proliferation and/or cell differentiation. Myosatellite cells can be identified based on their anatomical location between the basement membrane and the sarcolemma of muscle fibers. Myosatellite cells can also be identified based on the expression of markers, for example but not limited to, neural cell adhesion molecule (NCAM), CD56, Pax 3 or Pax 7. In one example, the myosatellite cell density of the hydrogel scaffold is, but is not limited to about 5×10⁵ cells/ml to 5×10⁶ cells/ml, about 5×10⁵ cells/ml to 9×10⁵ cells/ml, about 8×10⁵ cells/ml to 2×10⁶ cells/ml, about 1×10⁶ cells/ml to 5×10⁶ cells/ml, or about 5.0×10⁵ cells/ml, about 5.5×10⁵ cells/ml, about 6.0×10⁵ cells/ml, about 6.5×10⁵ cells/ml, about 7.0×10⁵ cells/ml, about 7.5×10⁵ cells/ml, about 8.0×10⁵ cells/ml, about 8.5×10⁵ cells/ml, about 9.0×10⁵ cells/ml, about 9.5×10⁵ cells/ml, about 1.0×10⁶ cells/ml, about 1.5×10⁶ cells/ml, about 2.0×10⁶ cells/ml, about 2.5×10⁶ cells/ml, about 3.0×10⁶ cells/ml, about 3.5×10⁶ cells/ml, about 4.0×10⁶ cells/ml, about 4.5×10⁶ cells/ml, about 5.0×10⁶ cells/ml. In another example, the hydrogel scaffold comprises 5×10⁵ cells/ml to 2×10⁶ cells/ml myosatellite cells. In another example, the hydrogel scaffold comprises 1×10⁶ cells/ml myosatellite cell.

As used herein, the term “myoblast” refers to a cell that is a progeny of a myosatellite cell. A myoblast is also a type of muscle progenitor cell, but is at a further stage of differentiation than a myosatellite cell. Myoblasts can proliferate and further differentiate into myofibers. Myoblasts can be identified based on the expression of markers, for example but not limited to, Pax 7, MyoD or myf-5. In one example, the myoblast density of the hydrogel scaffold is, but is not limited to about 5×10⁵ cells/ml to 5×10⁶ cells/ml, about 5×10⁵ cells/ml to 9×10⁵ cells/ml, about 8×10⁵ cells/ml to 2×10⁶ cells/ml, about 1×10⁶ cells/ml to 5×10⁶ cells/ml, or about 5.0×10⁵ cells/ml, about 5.5×10⁵ cells/ml, about 6.0×10⁵ cells/ml, about 6.5×10⁵ cells/ml, about 7.0×10⁵ cells/ml, about 7.5×10⁵ cells/ml, about 8.0×10⁵ cells/ml, about 8.5×10⁵ cells/ml, about 9.0×10⁵ cells/ml, about 9.5×10⁵ cells/ml, about 1.0×10⁶ cells/ml, about 1.5×10⁶ cells/ml, about 2.0×10⁶ cells/ml, about 2.5×10⁶ cells/ml, about 3.0×10⁶ cells/ml, about 3.5×10⁶ cells/ml, about 4.0×10⁶ cells/ml, about 4.5×10⁶ cells/ml, about 5.0×10⁶ cells/ml. In another example, the hydrogel scaffold comprises 5×10⁵ cells/ml to 2×10⁶ cells/ml myoblasts. In another example, the hydrogel scaffold comprises 1×10⁶ cells/ml myoblasts.

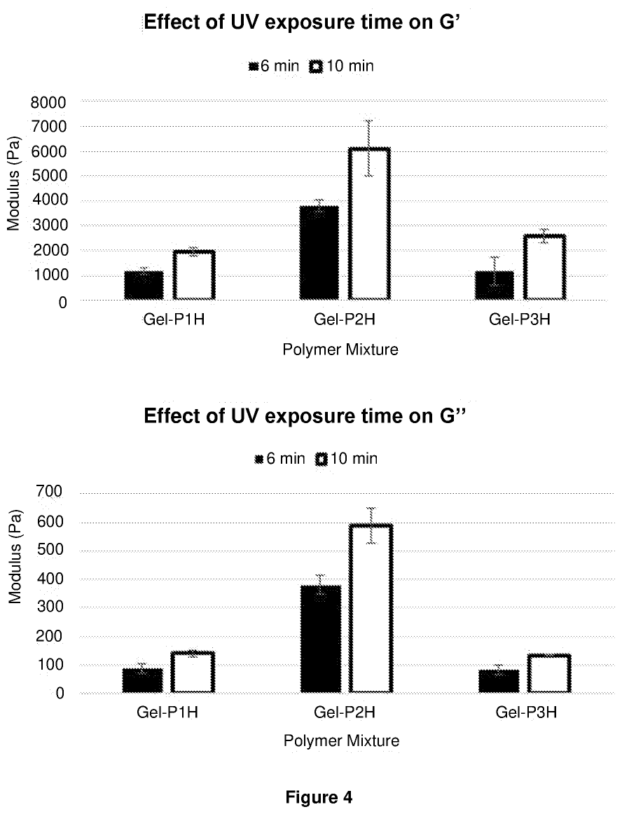

The hydrogel scaffolds of the present application provide higher resistance towards breakage, which is apparent from the loss modulus or storage modulus as shown in FIG. 4. Hydrogel scaffolds can have the following exemplary combinations. In one example, the hydrogel scaffold comprises: about 3.0 w/v % gelatin methacryloyl (GelMA); about 1.0 w/v % alginate; about 1.0 w/v % pectin; and one or more cell types. In another example, the hydrogel scaffold comprises: about 3.0 w/v % gelatin methacryloyl (GelMA); about 1.0 w/v % alginate; about 1.0 w/v % pectin; and one or more cell types, wherein the gelatin methacryloyl (GelMA) comprises a degree of methacryloyl substitution (DS) of about 50%. In another example, the hydrogel scaffold comprises: about 3.0 w/v % gelatin methacryloyl (GelMA); about 1.0 w/v % alginate; about 1.0 w/v % pectin; one or more cell types; and a storage modulus of about 6 kPa. In another example, the hydrogel scaffold comprises: about 3.0 w/v % gelatin methacryloyl (GelMA); about 1.0 w/v % alginate; about 1.0 w/v % pectin; one or more cell types; and a loss modulus of about 600 Pa.

In another example, the hydrogel scaffold comprises: about 3.0 w/v % gelatin methacryloyl (GelMA); about 1.0 w/v % alginate; about 1.0 w/v % pectin; and adipose-derived stem cells (ADSCs). In another example, the hydrogel scaffold comprises: about 3.0 w/v % gelatin methacryloyl (GelMA); about 1.0 w/v % alginate; about 1.0 w/v % pectin; and adipose-derived stem cells (ADSCs), wherein the gelatin methacryloyl (GelMA) comprises a degree of methacryloyl substitution (DS) of about 50%. In another example, the hydrogel scaffold comprises: about 3.0 w/v % gelatin methacryloyl (GelMA); about 1.0 w/v % alginate; about 1.0 w/v % pectin; adipose-derived stem cells (ADSCs); and a storage modulus of about 6 kPa. In another example, the hydrogel scaffold comprises: about 3.0 w/v % gelatin methacryloyl (GelMA); about 1.0 w/v % alginate; about 1.0 w/v % pectin; adipose-derived stem cells (ADSCs); and a loss modulus of about 600 Pa.

The hydrogel scaffold as disclosed herein have tunable properties for different applications by fine-tuning the different hydrogel formulations. By tailoring the formulations of the hydrogel scaffold as described herein, different mechanical strength can be achieved according to the need of the applications. The hydrogel scaffold of the present application can be cultured in a cell culture medium. In one example, the cell culture medium can comprise one or more growth factors. In another example, the cell culture medium can be absent of any growth factors. As used herein, the term “growth factor” refer to a protein that stimulates a variety of cellular processes including, but not limited to, cell proliferation, differentiation, migration or survival. Examples of growth factors include fibroblast growth factor (FGF), epidermal growth factor (EGF), hepatocyte growth factor (HGF), vascular endothelial growth factor (VEGF), transforming growth factor (TGF), insulin growth factor (IGF). Growth factors can also be categorized into more specific sub-categories for more specific uses. For example, a differentiation-inducing factor (DIF) is one such specific sub-category, and refers to a class of effector molecules that inhibits growth and promotes differentiation of cells. Some examples of differentiation-inducing factors can overlap with the growth factors. Examples of differentiation-inducing factors include fibroblast growth factor (FGF), epidermal growth factor (EGF), hepatocyte growth factor (HGF), vascular endothelial growth factor (VEGF), transforming growth factor (TGF), insulin growth factor (IGF). In one example, the methods as claimed herein do not need a DIF. Thus, in one example, the methods and compositions do not comprise a DIF. In one example, the methods and compositions do not comprise a one or more or all of the DIFs exemplified herein and listed above.

One example of the application of the hydrogel scaffold as disclosed herein is to allow the one or more cells to proliferate and/or differentiate into a specific lineage, in particular to promote myogenic differentiation. Present methods of promoting myogenic differentiation include the use of expensive differentiation media, which increases the cost. For example, previous studies have shown that myogenic differentiation of adipose-derived stem cells (ADSCs) require very specific media that includes combination of different or differentiation-inducing factors. Furthermore, the efficiency of using current methods for myogenic differentiation can be inefficient.

In view of the problem above, the inventors have also developed a method of myogenic differentiation using the hydrogel scaffold as disclosed herein, wherein complex cell culture media that requires different differentiation-inducing factors and supplements are not required. In one example, the present disclosure provides a method of myogenic differentiation comprising culturing one or more cell types in the hydrogel scaffold as disclosed herein in cell culture media, wherein the cell culture media comprises a basal medium and a serum. In one example, the present disclosure provides a method of myogenic differentiation comprising culturing one or more cell types in the hydrogel scaffold as disclosed herein in cell culture media, wherein the cell culture media comprises a basal medium and a serum, wherein the cell culture medium does not comprise a differentiation-inducing factor. As used herein, the term “basal medium” refers to a formulation that is essential for cell survival and growth. Basal medium is known in the art, and are media which comprises amino acids, glucose, and ions such as, but not limited to, calcium, magnesium, potassium, sodium, and phosphate). Basal media are known to be devoid of growth factors and/or differentiation-inducing factors. The basal medium can include but is not limited to Dulbecco's Modified Eagle Medium (DMEM), Advanced Dulbecco's Modified Eagle Medium (DMEM)/Ham's F12, DMEM/Ham's F12, Minimal Essential Medium (MEM), Knockout-DMEM (KO-DMEM), Glasgow Minimal Essential Medium (G-MEM), Basal Medium Eagle (BME), Iscove's Modified Dulbecco's Medium and Minimal Essential Medium (MEM), Ham's F-10, Ham's F-12, Medium 199, and RPMI 1640 Medium. In another example, the present disclosure provides a method of myogenic differentiation comprising culturing one or more cell types in the hydrogel scaffold as disclosed herein in the presence of a serum. In one example, the serum is fetal bovine serum (FBS) or horse serum. In a preferred example, the cell culture media comprises Dulbecco's Modified Eagle Medium (DMEM) and fetal bovine serum (FBS).

In another example, the cell culture media can further comprise a steroid or a hormone. In one example, the steroid is dexamethasone or hydrocortisone. In one example, the hormone is insulin.

The use of the cell culture media as disclosed herein, for example, a simple basal medium and a serum, is made possible by encapsulating the cells in the hydrogel during the production of the hydrogen scaffold, which provides physical cues to the cells. Without being bound by theory, the physical cues include, but are not limited to, tension. The cells that are encapsulated in the hydrogel undergo stretching during the production of the hydrogen scaffold, resulting in the cells being subjected to tension. This tension creates a 3D mechanotransduction that can drive cells to differentiate without the use of a differentiation media that comprises different differentiation-inducing factors. Therefore, the cells in the hydrogel scaffold as disclosed herein can undergo myogenic differentiation by culturing the hydrogel scaffold cell culture media comprises a basal medium and a serum. This method can be easily upscaled and therefore lead to increased production of artificial food.

In order to be able to produce the hydrogel scaffold, the method of fabricating the hydrogel scaffold has high tunability, high scalability and high encapsulation efficiency. Exemplary methods of fabricating the hydrogel scaffold include wet spinning. The method of fabricating the hydrogel scaffold as disclosed herein comprises for example:
a) preparing a solution comprising alginate and pectin;
b) treating the solution from step a) to a temperature of about 4-120° C.;
c) adding gelatin methacryloyl (GelMA) to the heat-treated solution from step b);
d) adding one or more cell types to solution from step c);
e) setting an injection rate of about 0.1-2.0 ml/min; and
f) wet-spinning a hydrogel scaffold.

The hydrogel scaffold is first fabricated by preparing a solution comprising alginate and pectin. In one example, the solution in step a) comprises about 0.5-2.0 w/v % alginate. In another example, the solution in step a) comprises, but is not limited to about 0.5-1.5 w/v % alginate, about 1.0-2.0 w/v % alginate, or about 0.5 w/v % alginate, about 0.6 w/v % alginate, about 0.7 w/v % alginate, about 0.8 w/v % alginate, about 0.9 w/v % alginate, about 1.0 w/v % alginate, about 1.1 w/v % alginate, about 1.2 w/v % alginate, about 1.3 w/v % alginate, about 1.4 w/v % alginate, about 1.5 w/v % alginate, about 1.6 w/v % alginate, about 1.7 w/v % alginate, about 1.8 w/v % alginate, about 1.9 w/v % alginate or about 2.0 w/v % alginate. In a preferred example, the solution in step a) comprises about 1.0 w/v % alginate.

In one example, the solution in step a) comprises about 0.5-2.0 w/v % pectin. In another example, the solution in step a) comprises, but is not limited to about 0.5-1.5 w/v % pectin, about 1.0-2.0 w/v % pectin, or about 0.5 w/v % pectin, about 0.6 w/v % pectin, about 0.7 w/v % pectin, about 0.8 w/v % pectin, about 0.9 w/v % pectin, about 1.0 w/v % pectin, about 1.1 w/v % pectin, about 1.2 w/v % pectin, about 1.3 w/v % pectin, about 1.4 w/v % pectin, about 1.5 w/v % pectin, about 1.6 w/v % pectin, about 1.7 w/v % pectin, about 1.8 w/v % pectin, about 1.9 w/v % pectin or about 2.0 w/v % pectin. In a preferred example, the solution in step a) comprises about 1.0 w/v % pectin.

After preparing the solution in step a), the solution is subjected to treatment at a set temperature. In one example, the temperature in step b) is about 4-120° C., or about 4-20° C., about 20-40° C., about 40-60° C., about 60-80° C., about 80-100° C., about 100-120° C., or about 4° C., about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., or about 120° C. In a preferred example, the temperature in step b) is about 80° C.

The solution from step a) is then subjected to treatment at a set temperature for a period of time. In one example, the solution from step a) can be treated for about 20-200 minutes, or about 20-60 minutes, about 50-90 minutes, about 80-120 minutes, about 110-150 minutes, about 140-180 minutes, about 170-200 minutes, or about 20 minutes, about 30 minutes, about 40 minutes, about 50 minutes, about 60 minutes, about 70 minutes, about 80 minutes, about 90 minutes, about 100 minutes, about 110 minutes, or about 120 minutes. In a preferred example, the solution from step a) is treated in step b) at the temperature of about 80° C. for about 60 minutes.

Next, gelatin methacryloyl (GelMA) is added to the heat-treated solution from step b). In one example, the solution in step c) comprises about 3.0-10.0 w/v % of gelatin methacryloyl (GelMA). In another example, the solution in step c) comprises, but is not limited to about 3.0-6.0 w/v % GelMA, about 5.0-8.0 w/v % GelMA, about 7.0-10.0 w/v % GelMA, or about 3.0 w/v % GelMA, about 4.0 w/v % GelMA, about 5.0 w/v % GelMA, about 6.0 w/v % GelMA, about 7.0 w/v % GelMA, about 8.0 w/v % GelMA, about 9.0 w/v % GelMA, or about 10.0 w/v % GelMA. In a preferred example, the solution in step c) comprises about 3.0 w/v % GelMA.

In one example, the GelMA of the solution in step c) comprises a degree of methacryloyl substitution (DS) of about 25-99%. In another example, the GelMA of the solution in step c) comprises a degree of methacryloyl substitution (DS) that can be, but is not limited to about 25%-50%, about 45%-70%, about 65%-99%, or about 25%-35%, about 35%-45%, about 45%-55%, about 55%-65%, about 65%-75%, about 75%-85%, about 85%-95%, about 90%-99%. In another example, the GelMA of the solution in step c) comprises a degree of methacryloyl substitution (DS) that can be, but is not limited to about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%. In a preferred example, the GelMA of the solution in step c) comprises a degree of methacryloyl substitution (DS) that is about 50%.

The solution from step c) can undergo a further treatment before it is subjected to step d). In one example, the solution from step c) is further incubated at a temperature of about 4-30° C., or about 4-10° C., about 10-20° C., about 20-30° C., or about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., In a preferred example, the solution from step c) is further incubated at a temperature of about 4° C.

The solution from step c) is further incubated overnight. In one example, the solution from step c) is further incubated for a period of about 12-24 hours, or about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In one example, the solution from step c) is further incubated at a temperature of about 4° C. for a period of about 12-24 hours. In a preferred example, the solution from step c) is further incubated at a temperature of about 4° C. for a period of about 16 hours. The overnight incubation of the solution from step c) allows complete hydration of GelMA and thus prevents the fabricated hydrogel scaffold from swelling.

In step d) one or more cell types are added to the solution from step c). In one example, the one or more cell types comprise stem cells and/or progenitor cells. In one example, the stem cells are added to the solution from step c) at a density of, but not limited to about 5×10⁵ cells/ml to 5×10⁶ cells/ml, about 5×10⁵ cells/ml to 9×10⁵ cells/ml, about 8×10⁵ cells/ml to 2×10⁶ cells/ml, about 1×10⁶ cells/ml to 5×10⁶ cells/ml, or about 5.0×10⁵ cells/ml, about 5.5×10⁵ cells/ml, about 6.0×10⁵ cells/ml, about 6.5×10⁵ cells/ml, about 7.0×10⁵ cells/ml, about 7.5×10⁵ cells/ml, about 8.0×10⁵ cells/ml, about 8.5×10⁵ cells/ml, about 9.0×10⁵ cells/ml, about 9.5×10⁵ cells/ml, about 1.0×10⁶ cells/ml, about 1.5×10⁶ cells/ml, about 2.0×10⁶ cells/ml, about 2.5×10⁶ cells/ml, about 3.0×10⁶ cells/ml, about 3.5×10⁶ cells/ml, about 4.0×10⁶ cells/ml, about 4.5×10⁶ cells/ml, about 5.0×10⁶ cells/ml. In another example, the stem cells are added to the solution from step c) at a density of 5×10⁵ cells/ml to 2×10⁶ cells/ml. In another example, the stem cells are added to the solution from step c) at a density of 1×10⁶ cells/ml.

In one example, the stem cells added to the solution from step c) are adipose-derived stem cells (ADSCs) or mesenchymal stem cells. In one example, the adipose-derived stem cells (ADSCs) or mesenchymal stem cells are added to the solution from step c) at a density of, but not limited to about 5×10⁵ cells/ml to 5×10⁶ cells/ml, about 5×10⁵ cells/ml to 9×10⁵ cells/ml, about 8×10⁵ cells/ml to 2×10⁶ cells/ml, about 1×10⁶ cells/ml to 5×10⁶ cells/ml, or about 5.0×10⁵ cells/ml, about 5.5×10⁵ cells/ml, about 6.0×10⁵ cells/ml, about 6.5×10⁵ cells/ml, about 7.0×10⁵ cells/ml, about 7.5×10⁵ cells/ml, about 8.0×10⁵ cells/ml, about 8.5×10⁵ cells/ml, about 9.0×10⁵ cells/ml, about 9.5×10⁵ cells/ml, about 1.0×10⁶ cells/ml, about 1.5×10⁶ cells/ml, about 2.0×10⁶ cells/ml, about 2.5×10⁶ cells/ml, about 3.0×10⁶ cells/ml, about 3.5×10⁶ cells/ml, about 4.0×10⁶ cells/ml, about 4.5×10⁶ cells/ml, about 5.0×10⁶ cells/ml. In another example, the adipose-derived stem cells (ADSCs) or mesenchymal stem cells are added to the solution from step c) at a density of 5×10⁵ cells/ml to 2×10⁶ cells/ml. In another example, the adipose-derived stem cells (ADSCs) or mesenchymal stem cells are added to the solution from step c) at a density of 1×10⁶ cells/ml.

In one example, the progenitor cells are added to the solution from step c) at a density of, but not limited to about 5×10⁵ cells/ml to 5×10⁶ cells/ml, about 5×10⁵ cells/ml to 9×10⁵ cells/ml, about 8×10⁵ cells/ml to 2×10⁶ cells/ml, about 1×10⁶ cells/ml to 5×10⁶ cells/ml, or about 5.0×10⁵ cells/ml, about 5.5×10⁵ cells/ml, about 6.0×10⁵ cells/ml, about 6.5×10⁵ cells/ml, about 7.0×10⁵ cells/ml, about 7.5×10⁵ cells/ml, about 8.0×10⁵ cells/ml, about 8.5×10⁵ cells/ml, about 9.0×10⁵ cells/ml, about 9.5×10⁵ cells/ml, about 1.0×10⁶ cells/ml, about 1.5×10⁶ cells/ml, about 2.0×10⁶ cells/ml, about 2.5×10⁶ cells/ml, about 3.0×10⁶ cells/ml, about 3.5×10⁶ cells/ml, about 4.0×10⁶ cells/ml, about 4.5×10⁶ cells/ml, about 5.0×10⁶ cells/ml. In another example, the progenitor cells are added to the solution from step c) at a density of 5×10⁵ cells/ml to 2×10⁶ cells/ml. In another example, the progenitor cells are added to the solution from step c) at a density of 1×10⁶ cells/ml.

In one example, the progenitor cells added to the solution from step c) are myoblasts or myosatellite cells. In one example, the myosatellite cells are added to the solution from step c) at a density of, but not limited to about 5×10⁵ cells/ml to 5×10⁶ cells/ml, about 5×10⁵ cells/ml to 9×10⁵ cells/ml, about 8×10⁵ cells/ml to 2×10⁶ cells/ml, about 1×10⁶ cells/ml to 5×10⁶ cells/ml, or about 5.0×10⁵ cells/ml, about 5.5×10⁵ cells/ml, about 6.0×10⁵ cells/ml, about 6.5×10⁵ cells/ml, about 7.0×10⁵ cells/ml, about 7.5×10⁵ cells/ml, about 8.0×10⁵ cells/ml, about 8.5×10⁵ cells/ml, about 9.0×10⁵ cells/ml, about 9.5×10⁵ cells/ml, about 1.0×10⁶ cells/ml, about 1.5×10⁶ cells/ml, about 2.0×10⁶ cells/ml, about 2.5×10⁶ cells/ml, about 3.0×10⁶ cells/ml, about 3.5×10⁶ cells/ml, about 4.0×10⁶ cells/ml, about 4.5×10⁶ cells/ml, about 5.0×10⁶ cells/ml. In another example, the myosatellite cells are added to the solution from step c) at a density of 5×10⁵ cells/ml to 2×10⁶ cells/ml. In another example, the myosatellite cells are added to the solution from step c) at a density of 1×10⁶ cells/ml.

In one example, the myoblast are added to the solution from step c) at a density of, but not limited to about 5×10⁵ cells/ml to 5×10⁶ cells/ml, about 5×10⁵ cells/ml to 9×10⁵ cells/ml, about 8×10⁵ cells/ml to 2×10⁶ cells/ml, about 1×10⁶ cells/ml to 5×10⁶ cells/ml, or about 5.0×10⁵ cells/ml, about 5.5×10⁵ cells/ml, about 6.0×10⁵ cells/ml, about 6.5×10⁵ cells/ml, about 7.0×10⁵ cells/ml, about 7.5×10⁵ cells/ml, about 8.0×10⁵ cells/ml, about 8.5×10⁵ cells/ml, about 9.0×10⁵ cells/ml, about 9.5×10⁵ cells/ml, about 1.0×10⁶ cells/ml, about 1.5×10⁶ cells/ml, about 2.0×10⁶ cells/ml, about 2.5×10⁶ cells/ml, about 3.0×10⁶ cells/ml, about 3.5×10⁶ cells/ml, about 4.0×10⁶ cells/ml, about 4.5×10⁶ cells/ml, about 5.0×10⁶ cells/ml. In another example, the myoblasts are added to the solution from step c) at a density of 5×10⁵ cells/ml to 2×10⁶ cells/ml. In another example, the myoblasts are added to the solution from step c) at a density of comprises 1×10⁶ cells/ml.

The solution from step d) further comprises a photoinitiator. As used herein, the term “photoinitiator” refers to a compound that creates reactive species, for example, but not limited to, free radicals, cations or anions when exposed to radiation such as ultraviolet. Photoinitiators are used to initiate a crosslinking or polymerization process upon exposure to radiation. Examples of photoinitiators include, but are not limited to, Irgacure 2959 or Lithium Phenyl (2,4,6-Trimethylbenzoyl) Phosphinate (LAP). In a preferred example, the photoinitiator is Irgacure 2959. In one example, Irgacure 2959 is comprised at about 0.5-2.0 w/v %, or about 0.5-1.5 w/v %, about 1.0-2.0 w/v %, or about 0.5 w/v %, about 0.6 w/v %, about 0.7 w/v %, about 0.8 w/v % pectin, about 0.9 w/v %, about 1.0 w/v %, about 1.1 w/v %, about 1.2 w/v %, about 1.3 w/v %, about 1.4 w/v %, about 1.5 w/v %, about 1.6 w/v %, about 1.7 w/v %, about 1.8 w/v %, about 1.9 w/v % or about 2.0 w/v %.

In step e), an injection rate needs to be set for wet spinning. In one example, the injection rate can be, but is not limited to, about 0.1-1.0 ml/min, about 1.0-2.0 ml/min, or about 0.1 ml/min, about 0.2 ml/min, about 0.3 ml/min, about 0.4 ml/min, about 0.5 ml/min, about 0.6 ml/min, about 0.7 ml/min, 0.8 ml/min, about 0.9 ml/min, about 1.0 ml/min, about 1.1 ml/min, about 1.2 ml/min, about 1.3 ml/min, about 1.4 ml/min, about 1.5 ml/min, about 1.6 ml/min, about 1.7 ml/min, 1.8 ml/min, about 1.9 ml/min, or about 2.0 ml/min. In a preferred example, injection rate in step e) is about 0.1 ml/min.

A needle must be used to facilitate the wet-spinning of a hydrogel scaffold is step f). In one example, the needle is a 25 G needle, or a 22 G needle, or a 16 G needle, or a 18 G needle. In a preferred example, a 25 G needle is used for step f).

The method of fabricating the hydrogel scaffold further comprises g) collecting the hydrogel scaffold a coagulation bath. The coagulation bath comprises CaCl₂). In one example, the concentration of CaCl₂) can be 0.5-2.0 w/v %. In another example, the concentration of CaCl₂) can be, but is not limited to about 0.5-1.5 w/v %, about 1.0-2.0 w/v %, or about 0.5 w/v %, about 0.6 w/v %, about 0.7 w/v %, about 0.8 w/v %, about 0.9 w/v %, about 1.0 w/v %, about 1.1 w/v %, about 1.2 w/v %, about 1.3 w/v %, about 1.4 w/v %, about 1.5 w/v %, about 1.6 w/v %, about 1.7 w/v %, about 1.8 w/v %, about 1.9 w/v % or about 2.0 w/v %. In a preferred example, the concentration of CaCl₂) is comprised at about 1.0 w/v %.

The hydrogel scaffold is left in the coagulation bath for about 1-10 minutes. In one example, the hydrogel scaffold is left in the coagulation bath for about 1-5 minutes, 5-10 minutes, or about 1 minute, about 2 minutes, about 3 minutes, about 4 minutes, about 5 minutes, about 6 minutes, about 7 minutes, about 8 minutes, about 9 minutes, or about 10 minutes. In a preferred example, the hydrogel scaffold is left in the coagulation bath for about 3 minutes.

In one example, the method of fabricating the hydrogel scaffold further comprises h) exposing the hydrogel scaffold from step g) to ultraviolet light.

In one example, the hydrogel scaffold from step g) is exposed to ultraviolet light for about 1-10 minutes. In one example, the hydrogel scaffold from step g) is exposed to ultraviolet light for about 1-5 minutes, 5-10 minutes, or about 1 minute, about 2 minutes, about 3 minutes, about 4 minutes, about 5 minutes, about 6 minutes, about 7 minutes, about 8 minutes, about 9 minutes, or about 10 minutes. In a preferred example, hydrogel scaffold from step g) is exposed to ultraviolet light for about 5 minutes.

In one example, the method of fabricating the hydrogel scaffold comprises:
a) preparing a solution comprising alginate and pectin;
b) treating the solution from step a) to a temperature of about 4-120° C.;
c) adding gelatin methacryloyl (GelMA) to the heat-treated solution from step b);
d) adding one or more cell types to solution from step c);
e) setting an injection rate of about 0.1-2.0 ml/min;
f) wet-spinning a hydrogel scaffold; and
g) collecting the hydrogel scaffold in a coagulation bath comprising CaCl₂).

In one example, the method of fabricating the hydrogel scaffold comprises:
a) preparing a solution comprising alginate and pectin;
b) treating the solution from step a) to a temperature of about 4-120° C.;
c) adding gelatin methacryloyl (GelMA) to the heat-treated solution from step b);
d) adding one or more cell types to solution from step c);
e) setting an injection rate of about 0.1-2.0 ml/min;
f) wet-spinning a hydrogel scaffold;
g) collecting the hydrogel scaffold in a coagulation bath comprising CaCl₂; and
h) exposing the hydrogel scaffold from step g) to ultraviolet light.

In another example, a method of fabricating a hydrogel scaffold comprises:
a) preparing a solution comprising alginate and pectin;
b) treating the solution from step a) to a temperature of about 4-120° C. for about 60 minutes;
c) adding about gelatin methacryloyl (GelMA) to the heat-treated solution from step b)
   wherein the solution from step c) is further incubated at a temperature of about 4° C. for about 12-24 hours;
d) adding one or more cell types to solution from step c),
   wherein the solution from step d) further comprises a photoinitiator;
e) setting an injection rate of about 0.1-2.0 ml/min;
f) wet-spinning a hydrogel scaffold, wherein a 25 G needle is used.

In another example, a method of fabricating a hydrogel scaffold comprises:
a) preparing a solution comprising alginate and pectin;
b) treating the solution from step a) to a temperature of about 4-120° C. for about 60 minutes;
c) adding about gelatin methacryloyl (GelMA) to the heat-treated solution from step b)
   wherein the solution from step c) is further incubated at a temperature of about 4° C. for about 12-24 hours;
d) adding one or more cell types to solution from step c),
   wherein the solution from step d) further comprises a photoinitiator;
e) setting an injection rate of about 0.1-2.0 ml/min;
f) wet-spinning a hydrogel scaffold, wherein a 25 G needle is used; and
g) collecting the hydrogel scaffold in a coagulation bath comprising CaCl₂).

In another example, a method of fabricating a hydrogel scaffold comprises:
a) preparing a solution comprising alginate and pectin;
b) treating the solution from step a) to a temperature of about 4-120° C. for about 60 minutes;
c) adding about gelatin methacryloyl (GelMA) to the heat-treated solution from step b)
   wherein the solution from step c) is further incubated at a temperature of about 4° C. for about 12-24 hours;
d) adding one or more cell types to solution from step c),
   wherein the solution from step d) further comprises a photoinitiator;
e) setting an injection rate of about 0.1-2.0 ml/min;
f) wet-spinning a hydrogel scaffold, wherein a 25 G needle is used;
g) collecting the hydrogel scaffold in a coagulation bath comprising CaCl₂); and
h) exposing the hydrogel scaffold from step g) to ultraviolet light.

In another example, a method of fabricating a hydrogel scaffold comprises:
a) preparing a solution comprising 0.5-2.0 w/v % alginate and 0.5-2.0 w/v % pectin;
b) treating the solution from step a) to a temperature of about 4-120° C. for about 60 minutes;
c) adding about 3.0-10.0 w/v % gelatin methacryloyl (GelMA) to the heat-treated solution from step b);
d) adding one or more cell types to solution from step c);
e) setting an injection rate of about 0.1-2.0 ml/min;
f) wet-spinning a hydrogel scaffold.

In another example, a method of fabricating a hydrogel scaffold comprises:
a) preparing a solution comprising 0.5-2.0 w/v % alginate and 0.5-2.0 w/v % pectin;
b) treating the solution from step a) to a temperature of about 4-120° C. for about 60 minutes;
c) adding about 3.0-10.0 w/v % gelatin methacryloyl (GelMA) to the heat-treated solution from step b);
d) adding one or more cell types to solution from step c);
e) setting an injection rate of about 0.1-2.0 ml/min;
f) wet-spinning a hydrogel scaffold; and
g) collecting the hydrogel scaffold in a coagulation bath comprising CaCl₂).

In another example, a method of fabricating a hydrogel scaffold comprises:
a) preparing a solution comprising 0.5-2.0 w/v % alginate and 0.5-2.0 w/v % pectin;
b) treating the solution from step a) to a temperature of about 4-120° C. for about 60 minutes;
c) adding about 3.0-10.0 w/v % gelatin methacryloyl (GelMA) to the heat-treated solution from step b);
d) adding one or more cell types to solution from step c);
e) setting an injection rate of about 0.1-2.0 ml/min;
f) wet-spinning a hydrogel scaffold;
g) collecting the hydrogel scaffold in a coagulation bath comprising CaCl₂; and
h) exposing the hydrogel scaffold from step g) to ultraviolet light.

In another example, a method of fabricating a hydrogel scaffold comprises:
a) preparing a solution comprising 1.0 w/v % alginate and 1.0 w/v % pectin;
b) treating the solution from step a) to a temperature of about 80° C. for about 60 minutes;
c) adding about 3.0 w/v % gelatin methacryloyl (GelMA) to the heat-treated solution from step b),
   wherein the solution from step c) is further incubated at a temperature of about 4° C. for about 16 hours;
d) adding one or more cell types to solution from step c),
   wherein the solution from step d) further comprises a photoinitiator;
e) setting an injection rate of about 0.1 ml/min;
f) wet-spinning a hydrogel scaffold.

The fabricated hydrogel scaffolds are collected, washed and transferred into cell culture.

As used in this application, the singular form “a,” “an,” and “the” include plural references unless the context clearly dictates otherwise. For example, the term “a genetic marker” includes a plurality of genetic markers, including mixtures and combinations thereof.

As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of concentration of a substance, size of a substance, length of time, or other stated values means+/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Material and Methods

Disclosed herein are methods to produce hydrogel scaffolds using hybrid technologies of utilizing wet-spun scaffold and stem cell differentiation by physical cues. The materials selected to fabricate scaffolds can be proteins from plants (soy proteins, zein and wheat gluten); plant polysaccharides (alginate, cellulose, pectin and starch) or others such as mammals, birds, reptiles, amphibians, marine animals and microorganisms. The cell source can be, but is not limited to, embryonic stem (ES) cells or adult stem cells harvested from pork, poultry, ovine, or bovine.

### Stem Cells Preparation

Cells in hydrogel scaffolds were differentiated from porcine adipose derived stem cells. Stem cells were first harvested from animal adipose tissue, expansion, and storage ex vivo after characterizations which includes multilineages differentiation abilities, doubling time and viability. Adipose tissue is an important source of stem cells due to large and easily accessible in body reservoirs. Adipose derived stem cells (ADSCs) and bone marrow derived stem cells (BMSCs) are two common adult stem cells for myogenic differentiation, but ADSCs' yield and proliferative abilities are greater than those of BMSCs. Although the stem cells in this protocol are harvested from porcine adipose tissue, they can also be harvested from other species such as bovine and chicken adipose tissues as well.

### Hydrogen Scaffold Materials Preparation and Formation

When selecting polymer combinations during the synthesis of a hydrogel scaffold as disclosed herein, it is important to consider the effects the polymer combination has on stem cell proliferation and the ability for the stem cells to undergo myogenic differentiation. Pectin is used as a thickener to reduce the concentration of alginate, wherein the latter is not favorable for cell attachment. GelMA is used to modulate the degradation rate of the hydrogel scaffold spun through photo induced covalent crosslinking. The polymer concentrations were optimized to reduce the breakage and degradation of the spun hydrogel scaffold.

Prior to spinning, 2 w/v % of sodium alginate and 2w/v % Pectin (Sigma) were dissolved in phosphate-buffered saline (PBS) at 80° C. for 1 hour. Separately, 6 w/v % of freeze-dried GelMA with 50% degree of substitution and 1.0 w/v % of Irgacure 2959 were dissolved in PBS at 37° C. for 30 min. The two solutions were then homogenized in a 1:1 ratio and stored overnight at 4° C. The pre-hydrogel solution therefore consists of 1% w/v of sodium alginate, 1% w/v of pectin, 3% w/v of GelMA and 0.5 w/v % of Irgacure 2959. The pre-gel solution will be thawed at 37° C. for at least 30 min prior to use.

The sterilization of pre-hydrogel solution was achieved via filtration with a 0.22 um syringe filter. To encapsulate cells, cells were resuspended in pre-hydrogel solution at a concentration of 1×10⁶ cells/mL.

In a sterile environment, the cell solution was wet-spun into a calcium chloride (1.0w/v %) coagulation bath. The hydrogel scaffold (microfibers) obtained were then placed under UV lamp (364 nm) for further crosslinking before they were washed with PBS thrice and transferred to well-plates for culture. The hydrogel scaffold were cultured in growth media and stained for DAPI (Sigma)—a nuclei stain, and monoclonal antimyosin (Sigma)—a specific marker for skeletal muscle cells on Day 9.

Hydrogel scaffold was extruded using wet spinning technology to scale up production. The hydrogel scaffold should possess desirable physical and biological properties such as adequate mechanical stiffness, precise surface microfeatures and surface ligands arrangement. These are critical factors to consider for scaffolds to service as physical cues in guiding stem cells encapsulated towards myogenesis effectively. The physical properties include stiffness, diameter, surface morphologies and degradation of hydrogel scaffold were characterized. In one example, the hydrogel scaffold is made of a mixture stem cells with polymers-plant proteins (soy proteins, zein and wheat gluten); plant polysaccharides (alginate, cellulose, pectin and starch) and GelMA. The hydrogel scaffold can be fabricated through wet spinning or 3D printing follow by crosslinking with coagulation buffer or ultraviolet light. In one example, the hydrogel scaffold can comprise microfibers which could be fabricated through electro-spinning follow by crosslinking with coagulation buffer or UV.

### Hydrogel Scaffold Culture and Characterization

After the hydrogel scaffold are spun, they are cultured in a cell culture medium to enable cell proliferation and differentiation. The cell viability, morphology, and differentiation of the cells encapsulated and cultured in the hydrogel scaffold were characterized using Live/Dead staining assay, F-actin staining and immunostaining for myogenic protein markers. For example, Yes-associated protein 1 (YAP1; hereafter referred to as YAP) was further evaluated to explore the influence of mechanical properties of the cell microenvironment.

Porcine Adipose Stem Cells (pADSCs) Extraction, Expansion and Differentiation

### Cell Extraction, Expansion and Storage

Subcutaneous adipose tissue was obtained by surgical procedures from adult male pigs, after anaesthesia and asepsis procedures. Subcutaneous fat (10 g per pig) was harvested from the dorsal abdominal area in mature female cross-bred domestic pigs between the age of 6 months to 1 year at a local abattoir, and then transported on ice in Dulbecco's modified Eagle medium (DMEM; Hyclone, Logan, Utah, USA) containing 200 U/ml of penicillin and 200 μg/ml of streptomycin (P/S; Gibco). The tissue was finely minced and washed with Dulbecco's phosphate-buffered saline (dPBS; Gibco, Grand Island, N.Y., USA) containing P/S. Each sample was homogenized with an equal volume of 0.1% collagenase type II (900 units of collagenase/1.5 ml DMEM/g fat) in DMEM with 200 U/ml of penicillin and 200 μg/ml of streptomycin (P/S; Gibco) in a shaking incubator rotating at 45 rpm at 37° C. for no more than 90 min. An equal volume (equal to the digestion medium) of culture medium containing DMEM/F12 with 10% fetal bovine serum (FBS) was added to stop the collagenase digestion. Digestion medium containing the digested adipose tissues was passed through a single layer of chiffon into a clean 250 ml serological bottle. The process can be accelerated be using a pair of forceps to depress the middle of the chiffon to provide a guide passage for the digestion media. The filtered digestion medium is centrifuged at 700×g for 10 min to collect the pellet of stromal-vascular cells. The supernatant is discarded without disturbing the pellets to remove the top layer of fat that contains mature adipocytes. The pellet is washed by adding 10 ml DMEM into each tube to resuspend the pellet with pipetting and gentle shaking of the tube, followed by centrifugation at 700×g for 6 min. The supernatant is decanted, and 10 ml of ACK lysis buffer is added to the pellet. The pellet is resuspended by pipetting and allowed to stand for 10 min at room temperature to lyse red blood cells in the stromal-vascular fraction. Equal amounts of DMEM (10 ml) is added to stop the reaction with gentle shaking of the tube, which is then centrifuged at 700×g for 10 min. The supernatant is discarded before 10 ml DMEM is added into each tube. The pellet is resuspended with repeated pipetting, and centrifuged at 700×g for 6 min. The DMEM with suspended cells is collected and passed through a 100 μm strainer into a new conical tube. The medium is pipetted several times, and aliquots of 20 μl of cell-containing medium is mixed with 180 μl of 0.4% trypan blue solution (1:10 dilution) in a new 1.5 ml Eppendorf tube. The cells are counted with a haemocytometer and the pADSCs are seeded at a density of 6,000 cells/cm² onto a desired size of culture dish or plate with culture medium containing DMEM/F12, 10% fetal bovine serum (FBS) with 200 U/ml of penicillin and 200 μg/ml of streptomycin (P/S; Gibco). The plates or dishes are incubated in the 37° C. incubator in air with 5% CO2 to allow cell attachment to the plates. On average, 10 grams of adipose tissue yielded 7 million cells at passage 0. Cells were expanded in culture until P4 and frozen in a solution with 10% dimethyl sulfoxide (DMSO; Sigma) in FBS, for long term storage (1×10⁶ cells/vial in a final volume of 1 mL, temp −196° C.). All the characterization assays were performed on pADSC cryopreserved for at least 3 months, and maximum cryopreserved up to 12 months.

### Cell Characterization

Multilineages Differentiation of pADSCs

At 70-90% confluence (following passage 4 for clones), cells were passaged by trypsinization and cultured in induction medium. Culture media were changed every 3-4 days throughout the study.

For adipogenesis, cells were cultured in adipocyte induction medium containing DMEM/F12 (with antibiotics of 1% Penicillin-Streptomycin (P/S)) containing 10 μg/ml insulin, 1 nM 3,3′,5-Triiodo-L-thyronine, 10 μg/ml transferrin, 1 μM dexamethasone and 1 μM rosiglitazone for 3 days and then medium was replaced by adipocyte maintenance medium with the same additions as the induction medium, but with omission of dexamethasone. Mature adipocytes were terminally differentiated by about 9 days. These adipocytes were ready for Oil Red O staining.

For chondrogenesis, cell pellets were cultured for the duration of differentiation in medium containing αMEM containing 1% FBS, 6.25 μg/ml insulin, 50 μg/ml ascorbate-2-phosphate, and 10 ng/ml transforming growth factor-β1 in a 15 ml tube. The chondrocyte induction medium was replaced every three days without removing cells on the bottom. Mature chondrocytes were formed in approximately 14 days. These chondrocytes were ready for Toluidine Blue O staining.

For osteogenesis, cells were cultured in induction medium containing DMEM with 10% FBS, 100 U/ml of penicillin and 100 μg/ml of streptomycin, 10 μm M β-glycerophosphate, 50 μg/ml ascorbate-2-phosphate and 10 μM dexamethasone. Osteocyte induction medium was changed every three days. Mature osteocytes will form by 14 days of differentiation. These osteocytes are ready for Alizarin Red S staining.

### Staining of Adipocytes, Osteocytes and Chondrocytes

### Oil Red O Staining for Adipocytes

At day 9, adipocyte maintenance medium was removed, and the cells were washed with PBS. The adipocytes were fixed in 4% paraformaldehyde solution for 10 min following washing with double-distilled water. After washing with double-distilled water, 60% isopropanol solution was added to the cells and incubated for 5 min. Isopropanol was replaced with 0.22 um syringe filtered 60% Oil Red O solution, wherein the Oil Red O solution is dissolved in isopropanol and diluted in deionized water. The cells are incubated for 15 min at room temperature. The Oil Red O solution is removed and washed with double-distilled water. The stained lipid droplets inside of adipocytes were ready for observation by light microscopy (FIG. 16).

### Alizarin Red S Staining for Osteocytes

At day 14, osteocyte induction medium was removed and the cells were washed with PBS. The osteocytes were fixed in 4% paraformaldehyde solution for 10 min following washing with double-distilled water. Cells were stained with 2% Alizarin Red S solution (pH=4.1-4.3) for 15 min on a gentle rocker shaker (100 rpm). After washing with double-distilled water, 10% formalin solution was added to the cells. The osteocytes were ready for observation by light microscopy (FIG. 16).

### Toluidine Blue O Staining for Chondrocytes

At day 14, cells pellets were washed with PBS and fixed with 4% paraformaldehyde solution for 10 min. After washing with double-distilled water, the cell pellets were embedded in optimal cutting temperature (OCT) compound and sectioned with a cryostat at a thickness of 5-10 m. The cryostat sections were stained with Toluidine Blue O solution (0.1% with pH 4.1) followed by washing with double-distilled water twice. The chondrocytes on the slides are ready for observation by light microscopy (FIG. 16).

### Experimental Results

pADSCs Morphology

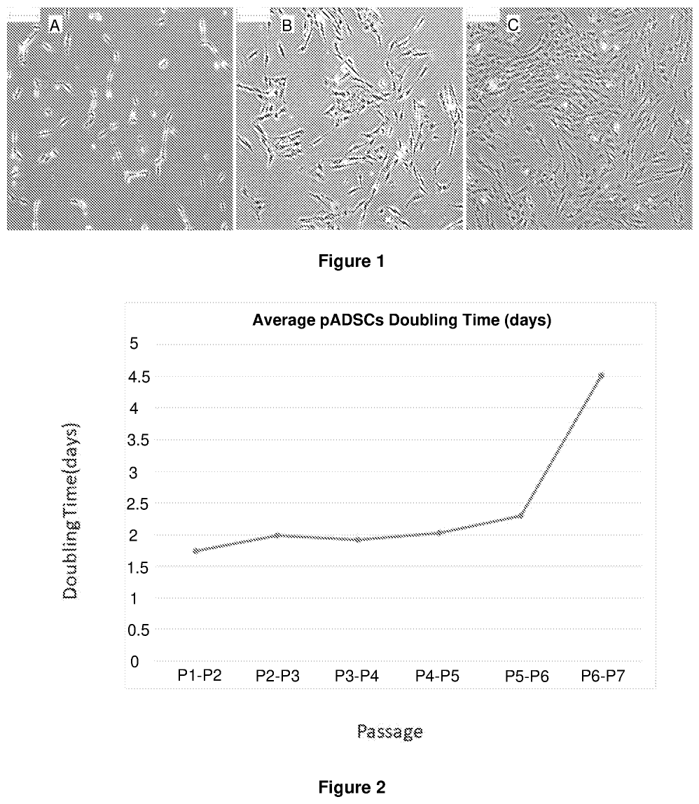

The porcine adipose-derived stem cells pADSCs derived from pig dorsal subcutaneous fat were seeded on the culture flask and shown in FIG. 1. The morphology of the pADSCs derived from the stromal-vascular fraction was similar to mouse or human ADSC. Day 3 after seeding with a seeding density of 6,000 cells/cm², pADSCs adhered and have an expanded fibroblast-like morphology (FIG. 1A). The pADSCs proliferated and spread on the surface of culture flask after 6 and 8-days culture (FIGS. 1B and C).

pADSC Proliferation Doubling Time

The pADSC's in vitro proliferation rate was examined through proliferation doubling time (DT) measurement. The DT value was determined for the studied cells. Proliferation doubling time (PDT) was calculated using the formula DT=T ln2/ln (Xₑ/X_{b}), in which T is the incubation time in hours, X_{b} represents the cell number at the beginning of the incubation time and Xₑ corresponds to the cell number at the end of incubation time (FIG. 2).

### Example 1—Scaffolds Materials Preparation

To fabricate hydrogel scaffolds for stem cell culture, there are a few requirements during polymers selection such as sterility, processability and biocompatibility. Sterility is required for cell culture to form the hydrogel scaffolds that can encapsulate cells. The polymer solution is thus sterilized before it is mixed with cells for wet spinning.

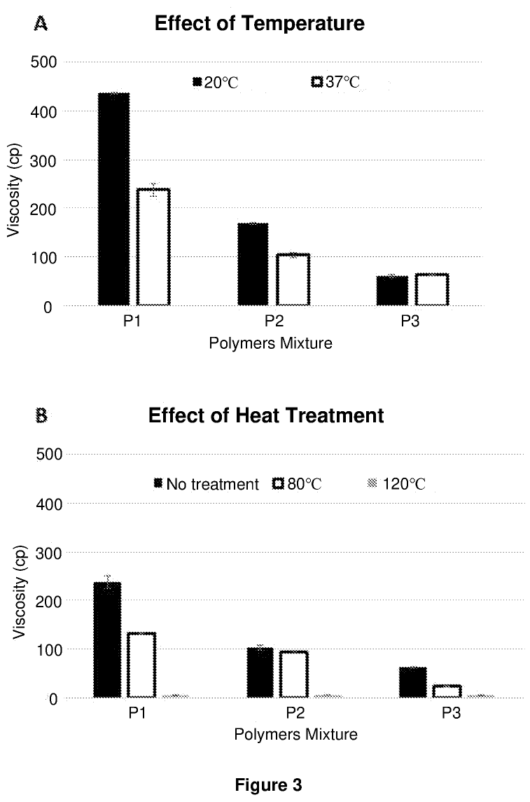

Filtration is the most suitable method to sterilize polymer solutions as both autoclave and irradiation may affect the molecular structures of polymers. However, polymer solutions could not pass through filters (0=0.2 μm) if it is too viscous. The viscosities of different polymers solutions at different temperatures were shown in FIG. 3A. It was observed that there is difficulty for the polymer solution to pass through filters when its viscosity is above 150 CP. To reduce the viscosity of polymer solution, heat treatment was performed for alginate and pectin before the solution was mixed with GelMA and cells. The effect of heat treatment on viscosities of polymers solutions were shown in FIG. 3B.

It was found that both heat treatments (80° C. for 2 hours and 120° C. for 20 mins) reduce the viscosity of polymer solutions, thus allowing them (P1, P2 and P3) to pass through filters for sterilization at 37° C. However, hydrogels formed after heat treatment (120° C. for 20 mins) resulted in fast degradation of hydrogel, therefore the rigidity of hydrogels could not be maintained for more than two weeks in culture medium, which is the minimum requirement for cell culture. The reason why heat treatment could reduce the viscosities of polymer solutions might be due to irreversible change of molecular conformation of polymers or degradation after heat treatment.

The mechanical properties of hydrogels formed by above polymer solutions were also evaluated. These polymer solutions were mixed with 0.5% Irgacure 2959 and then coagulated in 1% CaCl₂ buffer for 3 mins and then photo crosslinked under UV for 6 or 10 mins followed by washing with PBS, which is the same method used to form fibre like scaffolds through wet spinning. The results were shown in FIG. 4.

Both G′ and G″ of hydrogels formed by polymer solutions (P1H, P2H and P3H) could be influenced by UV exposure time. Long exposure time increased storage modulus (G′) and loss modulus (G″) of hydrogels. Substrate stiffness will drive the stem cells cultured on it to specific linages differentiation in 2D culture system. A substrate with an elastic modulus ranging from 8-17 KPa will induce stem cells myogenic differentiation. However, it may not so simple for 3D culture system and matrix plasticity also play an important role on cell behaviours.

### Example 2—Wet Spinning of Hydrogel Scaffold Encapsulated with Cells

The wet spinning process involves hydrogel scaffold extrusion into a solution. It is a voltage-free and could yield individual, dimensionally controllable hydrogel scaffold. Since it is a gentle process, applying lower temperatures has been the preferred method for production of hydrogel scaffold that cannot be melt spun. The wet spinning process offers the advantage of producing a wide variety of hydrogel scaffold cross-sectional shapes and sizes. Higher production speeds have been successfully attained in wet spinning over the last few years. This technology is used to form hydrogel scaffold encapsulated with cells.

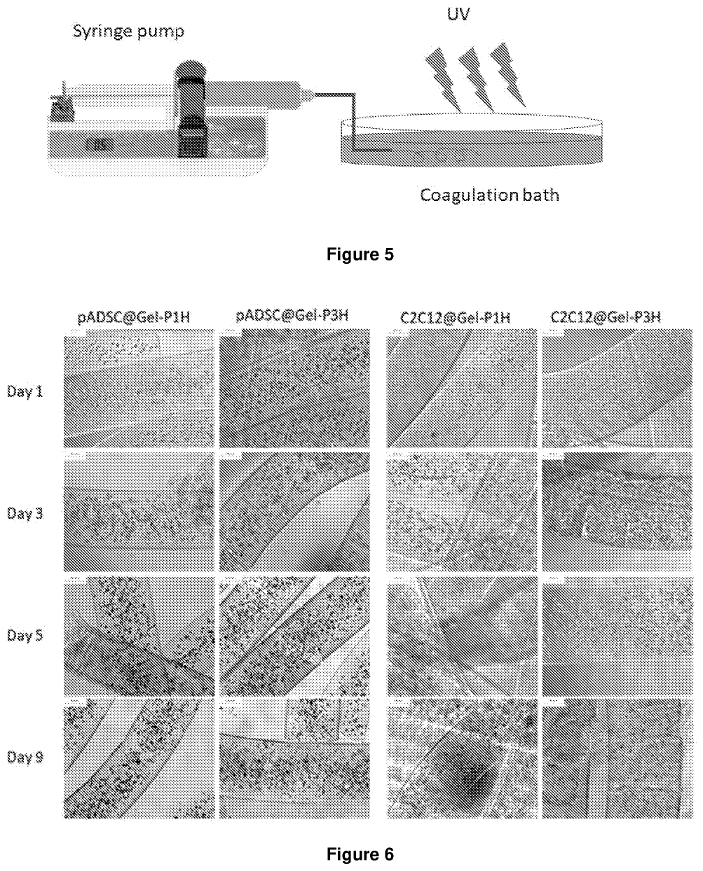

To spin the hydrogel scaffold, polymer solutions mixed with stem cells are loaded in a syringe with a 25 G needle, and injected into coagulation buffer with an injection rate which could give a uniform contours hydrogel scaffold (0.2 ml/min). An example of such a setup is shown in FIG. 5. When the hydrogel scaffolds are spun inside the coagulation buffer (1% CaCl₂) and 0.5% Irgacure 2959), alginate and pectin were crosslinked to maintain the hydrogel scaffold shape temporally. Subsequently, hydrogel scaffolds are exposed to UV (λ=365 nm) for 6 min followed by washing with PBS twice. Finally, the hydrogel scaffolds are transferred into culture plate with medium for cell proliferation and differentiation.

The morphologies of the spun hydrogel scaffolds with cells were evaluated through phase contrast microscopy as shown in FIG. 6. The diameters of the spun hydrogel scaffold using the above conditions were between 400-600 μm. The geometries and structures of these hydrogel scaffold were maintained for a 9-days culture period. Cells were spherical at the beginning of the culture period, and some of these cells started to spread on day 3 after the hydrogel scaffold are spun.

Two types of cells (C2C12 and pADSC) were selected, wherein each type of cell was mixed with hydrogel scaffolds made from either P1 or P3 polymer solutions. Cells displayed similar morphologies in both hydrogel scaffolds. More cells were also shown to be spread out on day 9 in comparison to the earlier time points.

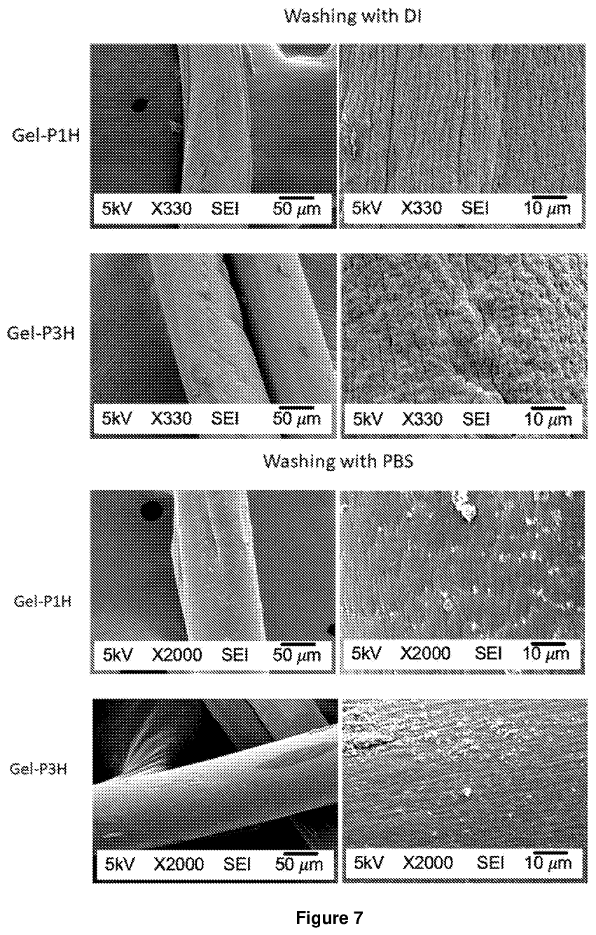

The surface morphologies of hydrogel scaffold were also examined using scanning electron microscopy (SEM), thereby providing more information about the surface microstructure of the hydrogel scaffolds. From FIG. 7, some precipitations were found on the surface of the hydrogel scaffold. It was assumed that was resulted from the reaction between PBS with Ca²⁺. PBS may remove Ca²⁺ from alginate gels and therefore created porous surface on the hydrogel scaffolds. In addition, there were aligned micro-grooves on the surface of the hydrogel scaffolds regardless washing with phosphate-buffered saline (PBS) or deionized water (DI).

### Example 3—Characterization of Cells Cultured in the Hydrogel Scaffolds

The growth of the cells encapsulated in the hydrogel scaffolds spun using wet spinning was studied. In addition, cell morphology, viability, and effects on differentiation was also evaluated. C2C12 is an immortalized mouse myoblast cell line that is capable of rapid proliferation under high serum conditions, as well as undergo differentiation into myotubes under low serum conditions. C2C12 cells can fuse to form multinucleated myotubes under low serum conditions, leading to the precursors of contractile skeletal muscle cells in the process of myogenesis. C2C12 is used here as a control to evaluate pADSCs growth and differentiation.

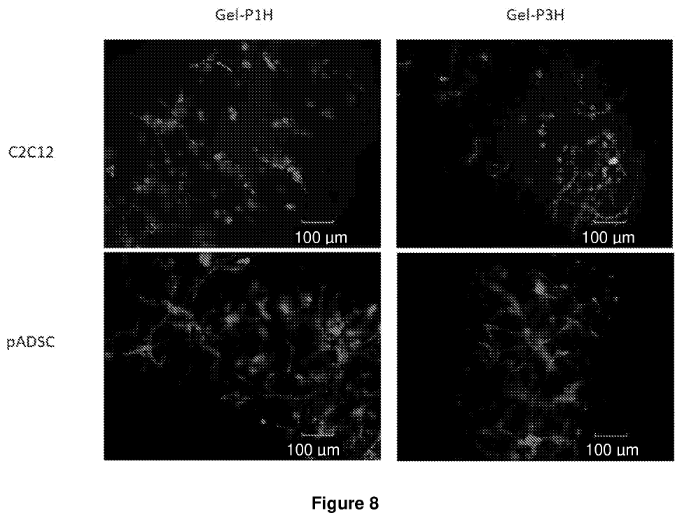

The viability of the ADSCs and C2C12 cultured in the hydrogel scaffolds was evaluated. A live-dead staining using calcein-AM and Ethidium Homodimer-1 (EthD-1) showed that both cells are still viable after culture in the hydrogel scaffolds post 9 days (FIG. 8).

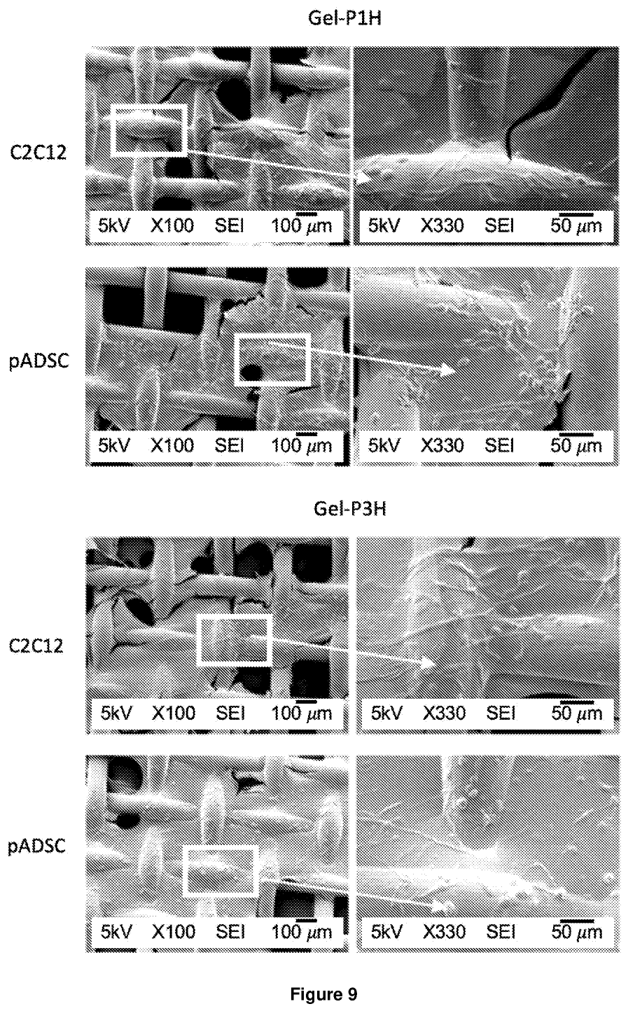

The morphologies of the ADSCs and C2C12 cultured in the hydrogel scaffolds were also observed using scanning electron microscope (SEM) (FIG. 9).

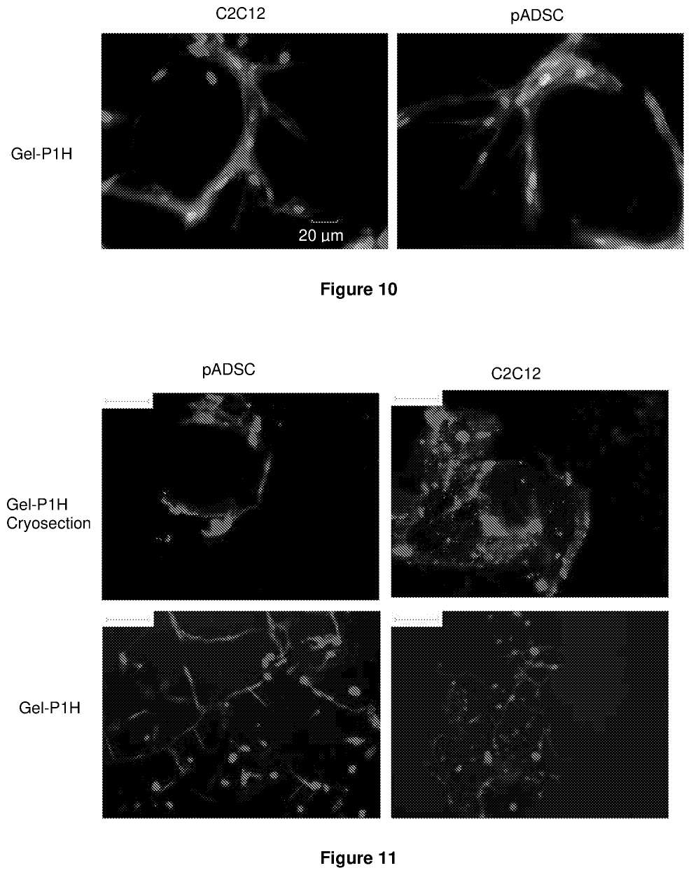

The morphologies of the cytoskeleton in the ADSCs and C2C12 cells cultured in the hydrogel scaffolds were also examined after 9 days culture using F-actin staining, and results were shown in FIG. 10. From these images, it was observed that some cells aligned “end to end” to form tubular structure, however it is not certain whether they fused to form multinucleated myotubes. In addition, there were still some cells that displayed spherical morphology.

Myogenic differentiation of the cells (ADSCs and C2C12) cultured in the hydrogel scaffolds was also evaluated after 9 days culture using immunostaining of monoclonal Anti-Myosin (Skeletal, Slow) and results were shown in FIG. 11. Monoclonal Anti-Myosin (Skeletal, Slow) recognizes an epitope located on the heavy meromyosin portion of human adult skeletal muscle slow myosin. It is highly specific for the slow myosin heavy chain of adult skeletal muscle and is thus used in the detection of Myosin.

The results shows that cells which have spread were positive for Myosin staining and revealed that both ADSCs and C2C12 were committed to myogenic differentiation. However, there were no striate patterns displayed, thereby suggesting that the differentiation was not mature.

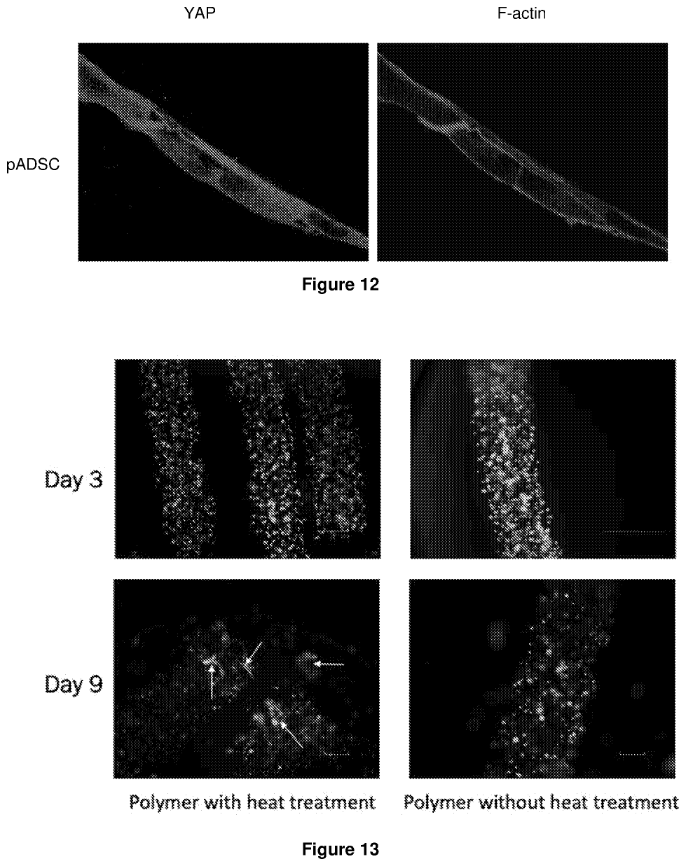

The yes-associated protein (YAP) is a mechanotransducer and can be used to detect a broad range of mechanical cues, from shear stress to cell shape and extracellular matrix rigidity, and translate them into cell-specific transcriptional programmes. It was previously reported that YAP is located inside the nucleus when cells were cultured on stiff substrate. However, the location of YAP during stem cells myogenesis is not known. YAP immunostaining for pADSCs is therefore done to understand the location of YAP during stem cells myogenesis (FIG. 12). From FIG. 12, YAP was still located in cytoplasm during cell myogenic differentiation process.

To spin the material without breakage, the range of components used are 0.5-2.0 w/v % of sodium alginate, 0.5-2.0 w/v % of pectin and 3.0-10.0 w/v % of GelMA with the 25-99% degree of substitution. The range of coagulation bath, calcium chloride, used to crosslink alginate and pectin was from 1.0-2.0 w/v %. The crosslinking time ranged between 1-10 min. The wavelength of UV used to crosslink GelMa was 365 nm and the irradiation time ranged from 5-10 min. The flow rate of the component injected into the coagulation bath ranged from 0.1-2.0 mL/min.

For the production of an exemplary performing hydrogel, a final concentration of 1.0 w/v % of sodium alginate, 1.0 w/v % of pectin, 3.0 w/v % of GelMa, with 50% degree of substitution, and 0.5 w/v % of Irgacure 2959 was injected into a coagulation bath, consisting of 1.0 w/v % of CaCl₂), at a flow rate of 0.1 mL/min. The material was left to crosslink in the coagulation bath for 3 min and was subsequently irradiated for GelMA crosslinking under a UV lamp with a wavelength of 365 nm for 5 min, followed by washing with PBS.

FIG. 13 shows C2C12 cells encapsulated within polymer which underwent heat treatment (left) and cells encapsulated within polymer which did not undergo heat treatment (right). It was observed that cells were stained green with calceinAM, indicative of live cells, implying that the polymer composition is suitable for the survival of cells. It was also observed that the cells encapsulated in the heat-treated polymer exhibited spreading while cells encapsulated in polymer that was not heat treated remained rounded.

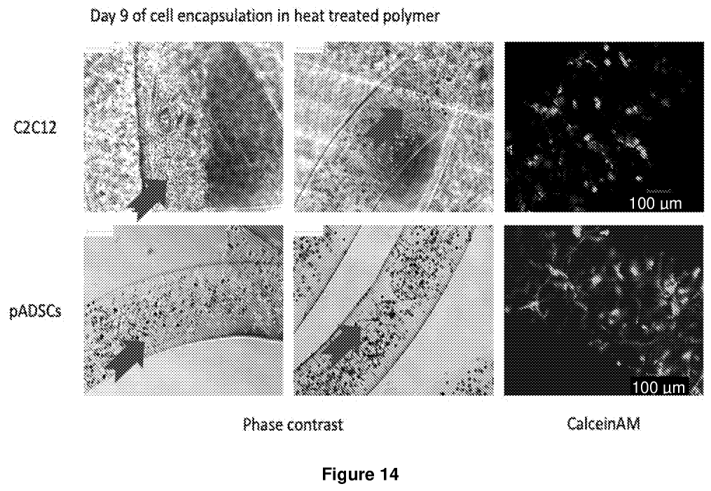

When porcine adipose derived stem cells (pADSCs) were encapsulated in the heat-treated polymer, similar to the observed phenomenon in C2C12, pADSCs also remain alive after 9 days of culture, and exhibited spreading as well (FIG. 14). In further experiments, it was also confirmed that these cells that exhibited spreading expressed myogenic markers, indicating that they were undergoing myogenic differentiation and/or myogenesis.

The cell density of pADSCs encapsulated within the hydrogel ranged between 5×10⁵ to 5×10⁶ cells/ml. The polymers were treated at various temperatures ranging from 4-120° C. and the treatment times ranged from 1-24 hours.

For sterilization, the materials were passed through a syringe filter with 0.22 um pore size. It was found that materials with viscosity above 150 cP cannot be passed through the 0.22 um syringe filter for sterilization.

The extrusion speed of the wet-spinning method ranged between 0.1 mL/min to 2.0 mL/min and the diameter of the wet-spun Hydrogel scaffolds produced ranged between 250-500 um.

Optimally, prior to spinning, alginate and pectin were dissolved and treated for 1 hr at 80° C. to so that the polymer strands can stretch and relax. GelMA was dissolved at 37° C. for 30 min and the final mixture was left to sit at 4° C. overnight for the complete hydration of GelMA and Irgacure 2959. Prior to wet-spinning, the solution was heated and to 37° C. The wet-spinning process for the best performing gel was described above and the diameter of hydrogel scaffolds obtained were approximately 300 um.

When hydrogel scaffolds were washed in PBS for 5 min, aligned microgrooves were present on the surface of the hydrogel scaffolds.

Normal growth media (DMEM+10% FBS+1% pen-strep) was used instead of skeletal muscle differentiation media during the differentiation process of porcine adipose-derived stem cells in wet-spun hydrogel scaffolds.

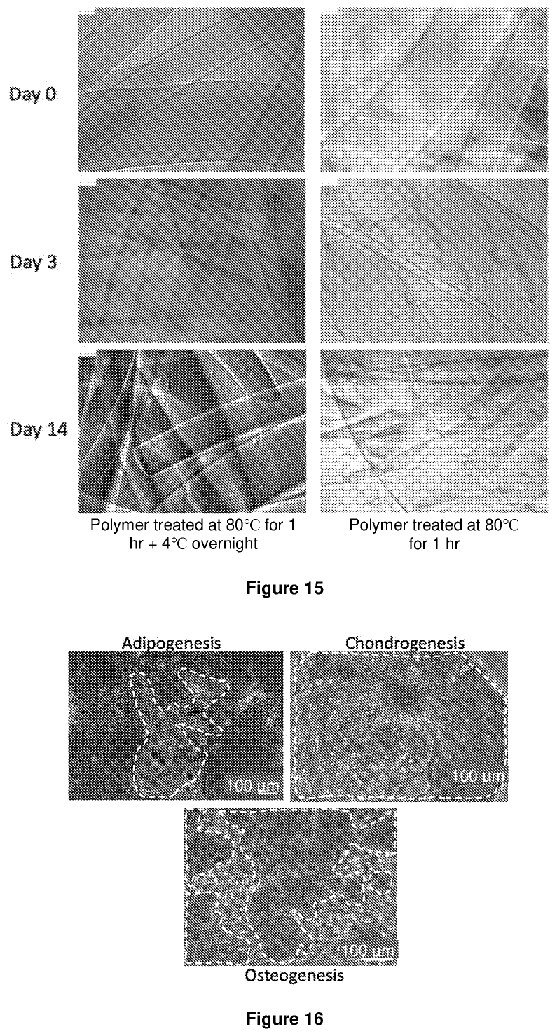

Experiments were performed to investigate the optimal treatment required for polymer solution prior to wet-spinning. As shown in FIGS. 3 and 4, the polymer viscosity changes based on the heat treatments ranging from 37° C.-120° C. Therefore, the effects of incubating polymer solutions at 4° C. overnight was investigated by comparing the wet-spun hydrogel scaffolds using polymer solutions with and without the 4° C. incubation overnight. It was observed that the hydrogel scaffolds spun with polymer solution without incubation at 4° C. overnight swelled significantly over 14 days (FIG. 15) as compared to the hydrogel scaffolds spun with polymer solution with incubation at 4° C. Therefore, incubation at 4° C. overnight was essential for complete hydration of GelMA.

### Commercial Applications of the Invention

The hydrogel scaffolds as disclosed herein does not break easily during the process of manufacturing, in comparison to other hydrogels with plant-derived components. In addition, the hydrogel scaffolds allow the encapsulated cells to spread within the hydrogel, which improves their growth. Using the hydrogel scaffolds as disclosed herein also allows stem cells or progenitor cells to undergo myogenic differentiation uses simple normal cell culture media such as DMEM and FBS, which is different from the conventional differentiation method which relies on the use of specific induction media or differentiation media, which are costly and not viable for upscaling production of the artificial food.

Furthermore, it is known that myogenic differentiation from ADSCs cannot be achieved from the simple cell culture media. By fabricating the hydrogel scaffolds with ADSCs encapsulated in them, the ADSCs in the hydrogel scaffold are subjected to physical cues such as tension, which enables the ADSCs in the hydrogel scaffold to undergo myogenic differentiation with just simple cell culture media such as DMEM and FBS. By switching to simple cell culture media that does not require any expensive chemical and/or biological components, this would make upscaling of cell production affordable.

The hydrogel scaffolds can be used in different applications such as, but not limited to, food production, medical application and cosmetic applications.

1. A hydrogel scaffold comprising:
about 3.0-10.0 w/v % gelatin methacryloyl (GelMA);
about 0.5-2.0 w/v % alginate;
about 0.5-2.0 w/v % pectin; and
one or more cell types.

2. The hydrogel scaffold of claim 1, wherein gelatin methacryloyl (GelMA) is comprised at about 3.0 w/v %.

3. The hydrogel scaffold of claim 1 or 2, wherein alginate is comprised at about 1.0 w/v %.

4. The hydrogel scaffold of any one of claims 1-3, wherein pectin is comprised at about 1.0 w/v %.

5. The hydrogel scaffold of any one of claims 1-4, wherein the one or more cell types comprise stem cells and/or progenitor cells.

6. The hydrogel scaffold of claim 5, wherein the stem cells are adipose-derived stem cells (ADSCs) or mesenchymal stem cells.

7. The hydrogel scaffold of claim 5, wherein the progenitor cells are myoblasts or myosatellite cells.

8. A method of myogenic differentiation comprising culturing one or more cell types in the hydrogel scaffold of any one of claims 1-7 in cell culture media, wherein the cell culture media comprises a basal medium and a serum.

9. The method of claim 8, wherein the basal medium is selected from a group consisting of Dulbecco's Modified Eagle Medium (DMEM), Advanced Dulbecco's Modified Eagle Medium (DMEM)/Ham's F12, DMEM/Ham's F12, Minimal Essential Medium (MEM), Knockout-DMEM (KO-DMEM), Glasgow Minimal Essential Medium (G-MEM), Basal Medium Eagle (BME), Iscove's Modified Dulbecco's Medium and Minimal Essential Medium (MEM), Ham's F-10, Ham's F-12, Medium 199, and RPMI 1640 Medium.

10. The method of claim 8, wherein the serum is fetal bovine serum (FBS) or horse serum.

11. The method of claims 8-10, wherein the cell culture media further comprises a steroid or a hormone.

12. A method of fabricating a hydrogel scaffold, the method comprising:
a) preparing a solution comprising alginate and pectin;
b) treating the solution from step a) to a temperature of about 4-120° C.;
c) adding gelatin methacryloyl (GelMA) to the heat-treated solution from step b);
d) adding one or more cell types to solution from step c);
e) setting an injection rate of about 0.1-2.0 ml/min;
f) wet-spinning a hydrogel scaffold.

13. The method of claim 12, wherein the solution in step a) comprises about 0.5-2.0 w/v % alginate.

14. The method of claim 12 or 13, wherein the solution in step a) comprises about 0.5-2.0 w/v % pectin.

15. The method of any one of claims 12-14, wherein the temperature in step b) is about 80° C.

16. The method of any one of claims 12-15, wherein the solution in step c) comprises about 3.0-10.0 w/v % of gelatin methacryloyl (GelMA).

17. The method of claim 12, wherein the injection rate in step e) is about 0.1 ml/min.
